SEQUENCE LISTING (3 sequences)

SEQ ID NO: 1 - protein, 140 aa, Homo sapiens
MDVFMKGLSKAKEGVVAAAEKTKQGVAEAAGKTKEGVLYVGSKTKEGVVHGVATVAEKTKEQVTNVGGAVVTGVTAVAQKTVEGAGSIAAATGFVKKDQLGKNEEGAPQEGILEDMPVDPDNEAYEMPSEEGYQDYEPEA

SEQ ID NO: 2 - protein, 140 aa, Homo sapiens
MDVFMKGLCKAKEGVVAAAEKTKQGVAEAAGKTKEGVLYVGCKTKEGVVHGVATVAEKTKEQVTNVGGCVVTGVTAVAQKTVEGAGSICAATGFVKKDQLGKNEEGAPQEGILEDMPVDPDNEAYEMPSEEGYQDYEPEA

SEQ ID NO: 3 - protein, 140 aa, Homo sapiens
MDVFMKGLCKAKEGVVAAAEKTKQGVAEAAGKTKEGVLYVGCKTKEGVVHGVATVAEKTKEQVTNVGGCVVTGVTAVAQKTVEGAGSICAATGFVKKDQLGKNEEGCPQEGILEDMPVDPDNECYEMPSEEGYQDYEPEA

(19) United States
(12) Patent Application Publication
(10) Pub. No.: US 2009/0169549 A1
(43) Pub. Date: Jul. 2, 2009

(54) CONFORMATIONAL ISOMERS OF ALPHA-SYNUCLEIN, ANTIBODIES THERETO AND METHODS OF THEIR MANUFACTURE AND USE

(75) Inventor: Jui-Yoa Chang, Houston, TX (US)

Correspondence Address: Pabst Patent Group LLP, 1545 PEACHTREE STREET NE, SUITE 320, ATLANTA, GA, 30309, US

(73) Assignee: The Board of Regents of the University of Texas System

(21) Appl. No.: 12/337,753

(22) Filed: Dec. 18, 2008

Related U.S. Application Data
(60) Provisional application No. 61/015,066, filed on Dec. 19, 2007.

(51) Int. Cl. A61K 39/395; A61K 38/17; C07K 14/435; C07K 16/18; G01N 33/566; C07K 1/00

(52) U.S. Cl. 424/133.1; 514/12; 530/350; 424/139.1; 530/387.3; 436/501; 530/408

(57) ABSTRACT

Conformational isomers of modified versions of α-Synuclein (αSyn), a protein that is associated with Parkinson's disease, have been designed and produced. These conformational isomers are produced by introducing cysteines into the α-Synuclein and scrambling the disulfide bonds to form stable and immunogenic isomers. These isomers are generically referred to as X-isomers. X-αSyn is an X-isomer of αSyn. X-αSyn is generally more immunogenic than wt-αSyn. Two groups of X-αSyn have been produced. One group is 3-disulfide X-αSyn(3SS) produced by introducing 6 Cys mutations into the αSyn. The second group is 2-disulfide X-αSyn(2SS), produced by introducing 4 Cys mutations into the α-Syn. In each of these two groups of X-αSyn, Cys was introduced not only wt-αSyn, but also in two Parkinson Disease associated αSyn mutants, A30P-αSyn and A53T-αSyn. All six sets of X-αSyn exhibit enhanced aggregation as compared to wt-αSyn, and should therefore more much more immunogenic. It was discovered that refolding the proteins in a CuSO₄ buffer greatly enhanced oxidation and therefore diversity of the refolded proteins. Production of antibodies to X-αSyn is useful because such antibodies should bind to wt-αSyn and thus be useful as a therapeutic or for detection of αSyn in subjects.

### CROSS-REFERENCE TO RELATED APPLICATIONS

This application claims benefit of and priority to U.S. Ser. No. 61/015,066 filed on Dec. 19, 2007.

### FIELD OF THE INVENTION

This invention relates to compositions and methods for diagnosing and treating diseases and disorders associated with α-synuclein, in particular to conformational isomers of α-synuclein, vaccines containing these and antibodies thereto, and methods of their manufacture and use.

### BACKGROUND OF THE INVENTION

Alpha-synuclein (αSyn) brain pathology is a conspicuous feature of several neurodegenerative diseases including Parkinson's disease (PD), dementia with Lewy bodies (DLB), the Lewy body variant of Alzheimer's disease (LBVAD), multiple systems atrophy (MSA), and neurodegeneration with brain iron accumulation type-1 (NBIA-1). Common to all of these diseases, termed synucleinopathies, are proteinaceous insoluble inclusions in the neurons and the glia which are composed primarily of αSyn.

Lewy bodies and Lewy neurites are intraneuronal inclusions which are composed primarily of αSyn. Lewy bodies and Lewy neurites are the neuropathological hallmarks Parkinson's disease (PD). PD and other synucleinopathic diseases have been collectively referred to as Lewy body disease (LBD). LBD is characterized by degeneration of the dopaminergic system, motor alterations, cognitive impairment, and formation of Lewy bodies (LBs) (McKeith, et al., Neurology, 47:1113-24 (1996)). Other LBDs include diffuse Lewy body disease (DLBD), Lewy body variant of Alzheimer's disease (LBVAD), combined PD and Alzheimer's disease (AD), and multiple systems atrophy. Dementia with Lewy bodies (DLB) is a term coined to reconcile differences in the terminology of LBDs.

Disorders with LBs are a common cause for movement disorders and cognitive deterioration in the aging population (Galasko, et al., Arch. Neurol, 51:888-95 (1994)). Although their incidence continues to increase, creating a serious public health problem, to date these disorders are neither curable nor preventable and understanding the causes and pathogenesis of PD is critical towards developing new treatments (Tanner et al., Curr. Opin. Neurol., 13:427-30 (2000)). The cause for PD is controversial and multiple factors have been proposed to play a role, including various neurotoxins and genetic susceptibility factors.

In recent years, new hope for understanding the pathogenesis of PD has emerged. Specifically, several studies have shown that the synaptic protein αSyn plays a central role in PD pathogenesis. αSyn is a 140 amino acid protein, which is enriched in the presynaptic terminals of neurons and is the major fibrillar component of Lewy bodies, a pathological hallmark of Parkinson's disease (Maroteaux, et al., Mol. Brain. Res., 11:335-343 (1991); Spillantini, et al., Nature, 388:839-840 (1997)). Its function has not yet been well established, though it might be involved in the regulation of dopamine neurotransmission (Lotharius, et al., J. Biol. Chem., 277:38884-38894 (2002); Perez, et al., J. Neurosci., 22, 3090-3099 (2002)) and the participation in a number of cell signaling pathways. Mutations in the αSyn gene co-segregate with rare familial forms of parkinsonism (Kruger, et al., Nature Gen., 18:106-8 (1998); Polymeropoulos, et al., Science, 276:2045-7 (1997)). Two different missense mutations in αSyn gene, corresponding to A53T and A30P substitutions in αSyn, have been identified to be associated with familial early-onset PD (Polymeropoulos, et al., Science, 276:2045-2047 (1997); Kruger, et al. Nat. Genet., 18:106-108 (1998)). Recently, a new mutation of E46K has been found to be involved in the pathogenesis of PD and Lewy body dementia (Zarranz, et al., Ann Neurol., 55:164-173 (2004)). Furthermore, the production of wild-type αSyn in transgenic mice (Masliah, et al. Science, 287:1265-1269 (2000)) or of the WT, A30P, and A53T in transgenic flies (Feany, et al., Nature, 404:394-398 (2000)) leads to the motor deficits and neuronal inclusions reminiscent of PD. Most recently, Singleton, et al. reported that locus triplication in the αSyn causes PD (Singleton, et al., Science, 302:841 (2003)). All these findings indicate that αSyn plays a critical role in the pathogenesis of PD and several other neurodegenerative synucleinopathies.

Protein conformation-dependent toxicity is an emerging theme in neurodegenerative disorders including the α-synucleinopathies (Muchowski, Neuron, 35:9-12 (2002); Uversky, et al. FEBS Lett., 500:105-108 (2001)). The native proteins are generally not pathogenic, however, they are the pool to provide the alternative conformations (normative isomers), which are the real culprit. Under different stressful conditions, the shift of equilibrium from the benign to the malignant isomers is commonly believed to be the underlying cause of these diseases. In this context, the more stable the native state is, the lower the risk of conformational change induced occurrence of diseases. However, genetic factors (mutations), environmental factors (pesticides) (Uversky, et al., FEBS Letters, 500:105-8 (2001)), and aging might all contribute to the destabilization the native state of αSyn and the initiation of the process of α-synucleinopathies.

Structurally, αSyn is intrinsically unstructured in its native state, which means that in the physiological conditions it lacks an ordered secondary structure (Weinreb, Biochemistry, 35:13709-13715 (1996)). Thermodynamically, the natively unfolded wild-type αSyn (wt-αSyn) must comprise a mixture of heterogeneous conformational isomers that exist in state of equilibrium. In this equilibrium model, it is generally believed that any shift of the equilibrium toward isomers of αSyn with highly aggregative propensity would trigger or initiate the process of synucleinopathies.

It would be advantageous to provide immunogenic compositions containing diverse and stabilized conformational isomers of αSyn that can provide many potential antigenic epitopes of αSyn for both B-cell and T-cell activation.

It would also be advantageous to provide immunogenic compositions containing diverse and stabilized conformational isomers of αSyn that are more immunogenic than native αSyn.

It would also be advantageous to provide antibodies produced in response to immunogenic compositions containing stabilized conformational isomers of αSyn.

It is therefore an object of the invention to provide immunogenic compositions containing diverse and stabilized conformational isomers of αSyn.

It is another object of the invention to provide immunogenic compositions of αSyn that are more immunogenic than native α-synuclein.

It is a further object of the invention to provide antibodies that bind to conformational isomers of αSyn.

It is an even further object of the invention to provide methods for treating or preventing synucleinopathies by administering vaccines containing one or more conformational isomers of αSyn.

It is another object of the invention to provide methods for treating or preventing synucleinopathies by administering antibodies that bind to one or more conformational isomers of αSyn.

It is yet another object of the invention to provide methods for diagnosing synucleinopathies by contacting samples with antibodies that bind to one or more conformational isomers of αSyn.

### SUMMARY OF THE INVENTION

Conformational isomers of modified versions of α-Synuclein (αSyn), a protein that is associated with Parkinson's disease, have been designed and produced. These conformational isomers are produced by introducing cysteines into a -Synuclein and scrambling the disulfide bonds to form stable and immunogenic isomers. These isomers are generically referred to as X-isomers. X-α-Synuclein (or X-αSyn) is an X-isomer of αSyn. X-αSyn is generally more immunogenic than natural a -Synuclein. Two groups of X-αSyn have been produced. One group is 3-disulfide X-αSyn (3SS) produced by introducing 6 Cys mutations into the αSyn. The second group is 2-disulfide X-αSyn (2SS), produced by introducing 4 Cys mutations into the αSyn. In each of these two groups of X-αSyn, Cys was introduced not only in wild-type (wt) αSyn, but also in two Parkinson Disease associated αSyn mutants, A30P-αSyn and A53T-αSyn:

X-wt-αSyn(3SS), X-A30P-αSyn(3SS), X-A53T-αSyn (3SS)

X-wt-αSyn(2SS), X-A30P-αSyn(2SS), X-A53T-αSyn (2SS)

All six sets of X-αSyn exhibit enhanced aggregation as compared to native αSyn, and should therefore more much more immunogenic. It was discovered that refolding the proteins in a CuSO₄ buffer greatly enhanced oxidation and therefore diversity of the refolded proteins. Production of antibodies to X-αSyn is useful because such antibodies are expected to bind to natural αSyn and thus be useful as a therapeutic or for detection of αSyn in subjects.

### BRIEF DESCRIPTION OF THE DRAWINGS

FIGS. 1A, 1B and 1C are a series of HPLC patterns of wt-αSyn(2SS) (FIG. 1A), A30P-αSyn(2SS) (FIG. 1B) and A53T-αSyn(2SS) (FIG. 1C) following the process of expression and purification. Wt and A53T mutants were isolated as three major 2-disulfide isomers. A30P mutant was recovered as a mixture of 1- and 2-disulfide isomers. Isomers were reduced using DTT to convert all three αSyn mutants to form fully reduced wt-αSyn(4C), A30P-αSyn(4C) and A53T-αSyn(4C).

FIGS. 2A-F are a series of HPLC patterns showing the oxidative folding of wt-αSyn(4C), A30P-αSyn(4C) and A53T-αSyn(4C). HPLC traces of the three fully reduced αSyn mutants are shown in the top panels. The fully reduced αSyn mutants were allowed to refold in Tris-HCl buffer (0.1 M, pH 8.4) (FIGS. 2A, B and C) or in Tris-HCl buffer containing CuSO₄ (2 μM) (FIGS. 2D, E and F). The oxidizing isomers were trapped by sample acidification at 1, 4 and 8 hours and analyzed by HPLC.

FIGS. 3A and 3B show far-UV CD spectra of variants of αSyn. FIG. 3A shows the spectra for mutants measured in acetate buffer (pH 4.5). FIG. 3B shows the spectra for mutants of αSyn(2SS) were measured in phosphate buffer (pH 7.4). wt-αSyn is represented by open squares, wt-αSyn(4C) by closed squares, A30P-αSyn(4C) by closed circles, and A53T-αSyn(4C) by closed triangles. Data are presented as [0] (deg cm² dmol⁻¹) as a function of wavelength (nm). Each αSyn(2SS) comprises 3 isomers. Protein concentration was 0.1 mg/ml. Wt-αSyn was included as a control. Each spectrum was an average of 10 scans. The content of secondary structure was calculated using the Softsec software provided by Jasco Inc.

FIG. 4 is a bar graph showing fibrillation (Thio T fluorescence) of wt-αSyn and 3 isolated 2-disulfide isomers of wt-αSyn(2SS) at 0, 24, 72 and 168 hours. Experiments were carried out in the PBS at 37° C. The protein concentration was 65 μM. ThT fluorescence was excited at 450 nm, and the emission wavelength was 482 nm.

FIG. 5 is a bar graph showing fibrillation (Thio T fluorescence) of collective isomers of wt-αSyn(2SS), A30P-αSyn(2SS) and A53T-αSyn(2SS) at 0, 24, 72 and 168 hours. Experiments were carried out in the PBS at 37° C. The protein concentration was 65 μM. ThT fluorescence was excited at 450 nm, and the emission wavelength was 482 nm.

FIGS. 6A, 6B and 6C are a series of HPLC patterns showing the oxidative folding of wt-αSyn(4C) (FIG. 6A), A30P-αSyn(4C) (FIG. 6B) and A53T-αSyn(4C) (FIG. 6D). HPLC traces of the three αSyn mutants reduced with DTT are shown in the bottom panels. The fully reduced αSyn mutants were allowed to refold in Tris-HCl buffer containing CuSO₄ (2 μM) (top panels).

FIGS. 7A, 7B and 7C are a series of HPLC patterns showing the oxidative folding of wt-αSyn(6C) (FIG. 7A), A30P-αSyn(6C) (FIG. 7B) and A53T-αSyn(6C) (FIG. 7C). HPLC traces of the three αSyn mutants reduced with DTT are shown in the bottom panels. The fully reduced αSyn mutants were allowed to refold in Tris-HCl buffer containing CuSO₄ (2 μM) (top panels).

### DETAILED DESCRIPTION OF THE INVENTION

### I. Definitions

Terms defined herein have meanings as commonly understood by a person of ordinary skill in the art. Terms such as “a”, “an” and “the” are not intended to refer to only a singular entity, but include the general class of which a specific example may be used for illustration.

As used herein, the term “disulfide” is used to define the bond formed between a sulfhydryl group of, e.g., one cysteine amino acid reside, and a sulfhydryl group of, e.g., a second cysteine amino acid residue. The two cysteine residues bound together by a disulfide bond are referred to as a “cystine” residue. As used herein, the term “disulfide” is equivalent to and interchangeable with disulfide bond, disulfide bridge, disulfide crosslink, and all other applicable terms and phrases known and used by one of skill in the art.

As used herein, the term “conformation” is used to define the spatial arrangement of amino acid residues of a peptide or protein. The term “conformation” is equivalent to and interchangeable with tertiary structure, three-dimensional structure, spatial arrangement, and all other applicable terms and phrases known and used by one of skill in the art.

As used herein, the term “native disulfide” is used to defined a disulfide bond in a “native” or “wild-type” protein resulting from the pairing between a sulfhydryl group on, e.g., one cysteine residue with a sulfhydryl group on, e.g., a second cysteine residue, wherein such pairing is “native” or “wild-type” pairing of sulfhydryl groups (i.e., native pairing of cysteine amino acid residues) and exists in the native conformation of a protein.

As used herein, the term “non-native disulfide” is used to define a disulfide bond in a protein resulting from the pairing between a sulfhydryl group on, e.g., one cysteine residue with a sulfhydryl group on, e.g., a second cysteine residue wherein such pairing is non-native pairing of sulfhydryl groups each or both resulting from the change or addition of; e.g., a cysteine at a location not associated with the “native” or “wild-type” amino acid sequence of the peptide or protein (i.e., non-native pairing of cysteine amino acid residues) and does not exist in the native conformation of a protein. Thus, a non-native disulfide bond may exist in scrambled disulfide isomers between a native and another native sulfhydryl group (but not between normally associated native or wild-type bonding residues), a native and a non-native sulfhydryl group or between two non-native sulfhydryl groups. Proteins that have non-native disulfide bonds may be further isolated and purified alone or in pools.

As used herein, “scrambled disulfide isomer” and “scrambled isomer” are used to define a conformational isomer of a native protein in which the scrambled isomer has at least one non-native disulfide, at least two non-native disulfides or more and the isomer has a non-native protein conformation. Combinations of scrambled isomers may also include native disulfides in addition to the at least one non-native disulfide. Each different species of scrambled disulfide isomer has a unique species-specific protein conformation and each of the species will differ from one another by at least one non-native disulfide. That is, each of the species of protein isomers includes at least one unique pairing of cysteine residues wherein the pairing is not found in the other species or in the native protein. The isomers may differ from one another and from the native protein by at least one, two or more non-native disulfide, e.g., at least two species-specific pairings of cysteine residues.

As used herein the term “isolated” is meant to describe a compound of interest (e.g., either a polynucleotide or a polypeptide) that is in an environment different from that in which the compound naturally occurs e.g. separated from its natural milieu such as by concentrating a peptide to a concentration at which it is not found in nature. “Isolated” is meant to include compounds that are within samples that are substantially enriched for the compound of interest and/or in which the compound of interest is partially or substantially purified.

As used herein, the term “polypeptide” refers to a chain of amino acids of any length, regardless of modification (e.g., phosphorylation or glycosylation).

As used herein, a “variant” polypeptide contains at least one amino acid sequence alteration as compared to the amino acid sequence of the corresponding wild-type polypeptide.

As used herein, an “amino acid sequence alteration” can be, for example, a substitution, a deletion, or an insertion of one or more amino acids.

As used herein, a “vector” is a replicon, such as a plasmid, phage, or cosmid, into which another DNA segment may be inserted so as to bring about the replication of the inserted segment. The vectors described herein can be expression vectors.

As used herein, an “expression vector” is a vector that includes one or more expression control sequences

As used herein, an “expression control sequence” is a DNA sequence that controls and regulates the transcription and/or translation of another DNA sequence.

As used herein, “operably linked” means incorporated into a genetic construct so that expression control sequences effectively control expression of a coding sequence of interest.

As used herein, a “fragment” of a polypeptide refers to any subset of the polypeptide that is a shorter polypeptide of the full length protein. Generally, fragments will be five or more amino acids in length.

As used herein, “conservative” amino acid substitutions are substitutions wherein the substituted amino acid has similar structural or chemical properties.

As used herein, “non-conservative” amino acid substitutions are those in which the charge, hydrophobicity, or bulk of the substituted amino acid is significantly altered.

As used herein, “isolated nucleic acid” refers to a nucleic acid that is separated from other nucleic acid molecules that are present in a mammalian genome, including nucleic acids that normally flank one or both sides of the nucleic acid in a mammalian genome.

As used herein with respect to nucleic acids, the term “isolated” includes any non-naturally-occurring nucleic acid sequence, since such non-naturally-occurring sequences are not found in nature and do not have immediately contiguous sequences in a naturally-occurring genome.

As used herein, the term “host cell” refers to prokaryotic and eukaryotic cells into which a recombinant expression vector can be introduced.

As used herein, “transformed” and “transfected” encompass the introduction of a nucleic acid (e.g. a vector) into a cell by a number of techniques known in the art.

The terms “individual”, “host”, “subject”, and “patient” are used interchangeably herein.

As used herein the term “effective amount” or “therapeutically effective amount” means a dosage sufficient to treat, inhibit, or alleviate one or more symptoms of a disease state, such as a Lewy body disease, being treated or to otherwise provide a desired pharmacologic and/or physiologic effect. The precise dosage will vary according to a variety of factors such as subject-dependent variables (e.g., age, immune system health, etc.), the disease, and the treatment being effected.

As used herein, the phrase that a molecule “specifically binds” to a target refers to a binding reaction which is determinative of the presence of the molecule in the presence of a heterogeneous population of other biologics. Thus, under designated immunoassay conditions, a specified molecule binds preferentially to a particular target and does not bind in a significant amount to other biologics present in the sample. Specific binding of an antibody to a target under such conditions requires the antibody be selected for its specificity to the target. A variety of immunoassay formats may be used to select antibodies specifically immunoreactive with a particular protein. For example, solid-phase ELISA immunoassays are routinely used to select monoclonal antibodies specifically immunoreactive with a protein. See, e.g., Harlow and Lane (1988) Antibodies, A Laboratory Manual, Cold Spring Harbor Publications, New York, for a description of immunoassay formats and conditions that can be used to determine specific immunoreactivity. Specific binding between two entities means an affinity of at least 10⁶, 10⁷, 10⁸, 10⁹, or 10¹⁰ M⁻¹. Affinities greater than 10⁸ M⁻¹ are preferred.

As used herein, the terms “antibody” or “immunoglobulin” are used to include intact antibodies and binding fragments thereof. Typically, fragments compete with the intact antibody from which they were derived for specific binding to an antigen fragment including separate heavy chains, light chains Fab, Fab′F(ab′)2, Fabc, and Fv. Fragments are produced by recombinant DNA techniques, or by enzymatic or chemical separation of intact immunoglobulins. The term “antibody” also includes one or more immunoglobulin chains that are chemically conjugated to, or expressed as, fusion proteins with other proteins. The term “antibody” also includes bispecific antibody. A bispecific or bifunctional antibody is an artificial hybrid antibody having two different heavy/light chain pairs and two different binding sites. Bispecific antibodies can be produced by a variety of methods including fission of hybridomas or linking of Fab′ fragments. See, e.g., Songsivilai & Lachmann, Clin. Exp. Immunol, 79:315-321 (1990); Kostelny, et al., J. Immunol., 148:1547-1553 (1992).

As used herein, an “antigen” is an entity to which an antibody specifically binds.

As used herein, the terms “epitope” or “antigenic determinant” refer to a site on an antigen to which B and/or T cells respond. B-cell epitopes can be formed both from contiguous amino acids or noncontiguous amino acids juxtaposed by tertiary folding of a protein. Epitopes formed from contiguous amino acids are typically retained on exposure to denaturing solvents whereas epitopes formed by tertiary folding are typically lost on treatment with denaturing solvents. An epitope typically includes at least 3, and more usually, at least 5 or 8-10 amino acids, in a unique spatial conformation. Methods of determining spatial conformation of epitopes include, for example, x-ray crystallography and 2-dimensional nuclear magnetic resonance. See, e.g., Epitope Mapping Protocols in Methods in Molecular Biology, Vol. 66, Glenn E. Morris, Ed. (1996). Antibodies that recognize the same epitope can be identified in a simple immunoassay showing the ability of one antibody to block the binding of another antibody to a target antigen. T-cells recognize continuous epitopes of about nine amino acids for CD8 cells or about 13-15 amino acids for CD4 cells. T cells that recognize the epitope can be identified by in vitro assays that measure antigen-dependent proliferation, as determined by ³H-thymidine incorporation by primed T cells in response to an epitope (Burke et al., J. Inf. Dis., 170:1110-19 (1994)), by antigen-dependent killing (cytotoxic T lymphocyte assay, Tigges, et al., J. Immunol., 156:3901-3910) or by cytokine secretion.

As used herein, the term “immunological” or “immune” response is the development of a beneficial humoral (antibody mediated) and/or a cellular (mediated by antigen-specific T cells or their secretion products) response directed against an amyloid peptide in a recipient patient. Such a response can be an active response induced by administration of immunogen or a passive response induced by administration of antibody or primed T-cells. A cellular immune response is elicited by the presentation of polypeptide epitopes in association with Class I or Class II MHC molecules to activate antigen-specific CD4⁺ T helper cells and/or CD8⁺ cytotoxic T cells. The response may also involve activation of monocytes, macrophages, NK cells, basophils, dendritic cells, astrocytes, microglia cells, eosinophils or other components of innate immunity. The presence of a cell-mediated immunological response can be determined by proliferation assays (CD4⁺ T cells) or CTL (cytotoxic T lymphocyte) assays. The relative contributions of humoral and cellular responses to the protective or therapeutic effect of an immunogen can be distinguished by separately isolating antibodies and T-cells from an immunized syngeneic animal and measuring protective or therapeutic effect in a second subject.

An “immunogenic agent” or “immunogen” is capable of inducing an immunological response against itself on administration to a mammal, optionally in conjunction with an adjuvant.

As used herein, the term “adjuvant” refers to a compound that when administered in conjunction with an antigen augments the immune response to the antigen, but when administered alone does not generate an immune response to the antigen. Adjuvants can augment an immune response by several mechanisms including lymphocyte recruitment, stimulation of B and/or T cells, and stimulation of macrophages.

### II. Compositions

A. Immunogenic Compositions

1. Conformational Isomers of α-Synuclein (αSyn)

The immunogenic compositions disclosed herein include one or more isolated and purified non-native, stabilized conformational isomers of αSyn. Stabilized conformational isomers of αSyn are generated through the technique of disulfide scrambling. This technique requires proteins that contain at least three cysteines, either naturally or by site specific substitution. In this technique, proteins are reduced and denatured in the presence of a reducing agent and a denaturant. After exclusion of the reductant and denaturant, the reduced and denatured proteins are allowed to refold in the presence of a redox buffer. The refolding proteins rearrange their disulfide bonds through a series of thiol-disulfide exchange reactions and can assume a number of heterogeneous disulfide isomer intermediates during oxidative folding that leads to the formation of the native structure. Conformational disulfide isomers of proteins are not interconvertible in the absence of a thiol catalyst or at acidic pH, and thus can be isolated during denaturation or refolding and separated as stable isomers by conventional separation techniques. As described below in the examples, it has been discovered that these proteins refold with greater degrees of oxidation in the presence of a CuSO₄ refolding buffer.

The amino acid sequence of wild-type human αSyn is provided by NCBI Accession No. NP_{—}000336. Human αSyn can have at least 80%, 85%, 90%, 95%, 99% or 100% sequence identity to:

| (SEQ ID NO:1) | |
|---|---|
| 1 | MDVFMKGLSK AKEGVVAAAE KTKQGVAEAA GKTKEGVLYV GSKTKEGVVH GVATVAEKTK |
| 61 | EQVTNVGGAV VTGVTAVAQK TVEGAGSIAA ATGFVKKDQL GKNEEGAPQE GILEDMPVDP |
| 121 | DNEAYEMPSE EGYQDYEPEA |

Native human αSyn does not contain any cysteine residues. However, stabilized conformational isomers of αSyn can be generated from αSyn polypeptides engineered to contain 3 or more cysteines. The αSyn polypeptides engineered to contain 3 or more cysteines can be of any species of origin. In one embodiment, the αSyn polypeptide is from a mammalian species. In a preferred embodiment the αSyn polypeptide is of human origin.

The αSyn polypeptides engineered to contain 3 or more cysteines can be “variant” polypeptides that contain at least one amino acid sequence alteration as compared to the amino acid sequence of the corresponding wild-type polypeptide. An amino acid sequence alteration can be, for example, a substitution, a deletion, or an insertion of one or more amino acids.

A variant αSyn polypeptide can have any combination of amino acid substitutions, deletions or insertions. In one embodiment, isolated αSyn variant polypeptides have an integer number of amino acid alterations such that their amino acid sequence shares at least 60, 70, 80, 85, 90, 95, 97, 98, 99, 99.5 or 100% identity with an amino acid sequence of a wild type αSyn polypeptide. In a preferred embodiment, αSyn variant polypeptides have an amino acid sequence sharing at least 60, 70, 80, 85, 90, 95, 97, 98, 99, 99.5 or 100% identity with the amino acid sequence of wild type human αSyn polypeptide.

Percent sequence identity can be calculated using computer programs or direct sequence comparison. Preferred computer program methods to determine identity between two sequences include, but are not limited to, the GCG program package, FASTA, BLASTP, and TBLASTN (see, e.g., D. W. Mount, 2001, Bioinformatics: Sequence and Genome Analysis, Cold Spring Harbor Laboratory Press, Cold Spring Harbor, N.Y.). The BLASTP and TBLASTN programs are publicly available from NCBI and other sources. The well-known Smith Waterman algorithm may also be used to determine identity.

Exemplary parameters for amino acid sequence comparison include the following: 1) algorithm from Needleman and Wunsch (J. Mol. Biol., 48:443-453 (1970)); 2) BLOSSUM62 comparison matrix from Hentikoff and Hentikoff (Proc. Natl. Acad. Sci. U.S.A., 89:10915-10919 (1992)) 3) gap penalty 12; and 4) gap length penalty 4. A program useful with these parameters is publicly available as the “gap” program (Genetics Computer Group, Madison, Wis.). The aforementioned parameters are the default parameters for polypeptide comparisons (with no penalty for end gaps).

Alternatively, polypeptide sequence identity can be calculated using the following equation: % identity=(the number of identical residues)/(alignment length in amino acid residues)*100. For this calculation, alignment length includes internal gaps but does not include terminal gaps.

Amino acid substitutions in αSyn polypeptides may be “conservative” or “non-conservative”. As used herein, “conservative” amino acid substitutions are substitutions wherein the substituted amino acid has similar structural or chemical properties, and “non-conservative” amino acid substitutions are those in which the charge, hydrophobicity, or bulk of the substituted amino acid is significantly altered. Non-conservative substitutions will differ more significantly in their effect on maintaining (a) the structure of the peptide backbone in the area of the substitution, for example, as a sheet or helical conformation, (b) the charge or hydrophobicity of the molecule at the target site, or (c) the bulk of the side chain.

In one embodiment the αSyn polypeptide engineered to contain 3 or more cysteines is human A30P-αSyn. In another embodiment the αSyn polypeptide engineered to contain 3 or more cysteines is human A53T-αSyn. As described in the examples, there are naturally occurring variants, such as those associated with Parkinson's Disease, that can be modified in the same manner as the wt-αSyn.

The αSyn polypeptides disclosed herein can be full-length polypeptides, or can be a fragment of full length αSyn polypeptides. As used herein, a fragment of αSyn refers to any subset of the polypeptide that is a shorter polypeptide of the full length protein.

Any amino acid at any position of αSyn may be mutated to a cysteine for production of conformational isomers that can be used as immogens. In preferred embodiments, amino acids with side groups similar to cysteine, such as alanine, serine and threonine, are mutated to cysteine.

The αSyn polypeptides useful to generate stabilized conformational isomers have at least 3 cysteines, but can contain any integer number of cysteines, including 3, 4, 5, 6, 7, 8, 9, 10, 11, 12, 13, 14, 15, 16, 17, 18, 19 or 20 cysteines. In one embodiment the αSyn polypeptides contain 4 cysteines. An exemplary wt-αSyn polypeptide containing 4 cysteines at positions 9, 42, 69 and 89, is provided below:

| (SEQ ID NO:2) | |
|---|---|
| 1 | MDVFMKGLCK AKEGVVAAAE KTKQGVAEAA GKTKEOVLYV GCKTKEGVVH GVATVAEKTK |
| 61 | EQVTNVGGCV VTGVTAVAQK TVEGAGSICA ATGFVKKDQL GKNEEGAPQE GILEDMPVDP |
| 121 | DNEAYEMPSE EGYQDYEPEA |

In another embodiment the αSyn polypeptides contain 6 cysteines. An exemplary wt-αSyn polypeptide containing 6 cysteines at positions 9, 42, 69, 89, 107 and 124 is provided below:

| (SEQ ID NO:3) | |
|---|---|
| 1 | MDVFMKGLCK AKEGVVAAAE KTKQGVAEAA GKTKEGVLYV GCKTKEGVVH GVATVAEKTK |
| 61 | EQVTNVGGCV VTGVTAVAQK TVEGAGSICA ATGFVKKDQL GKNEEGCPQE GILEDMPVDP |
| 121 | DNECYEMPSE EGYQDYEPEA |

Increasing the number of cysteine mutations in the αSyn polypeptides greatly increases the diversity of the stabilized conformational isomers that can be generated. For example, αSyn polypeptides containing 3, 4, 5, 6 and 7 cysteines can form 15, 105, 945, 10,395 and 135,135 possible disulfide isomers. The diversity of possible disulfide isomers is further enhanced by altering the location of the cysteines within the primary amino acid sequence. Thus, a huge diversity of stabilized conformational isomers can be generated by altering the number and location of cysteine residues throughout the αSyn amino acid sequence.

The immunogenic compositions may contain one or more stabilized conformational isomers of αSyn. In one embodiment, the immunogenic compositions contain a single conformational isomer of αSyn purified from other conformational isomers. In another embodiment, the immunogenic compositions contain multiple conformational isomers, or pools of conformational isomers of αSyn.

The diversity of conformational isomers of αSyn polypeptides can be further increased by altering the denaturing and/or refolding conditions, as discussed in more detail below. The conditions that can be altered are the denaturant, the thiol reducing reagent, redox agents that may be added to catalyze refolding, and the extent and time in which these conditions are applied.

2. Adjuvants

Optionally, the immunogenic compositions may include other excipients or adjuvants to enhance immunogenicity. The adjuvant can be, but is not limited to, one or more of the following: oil emulsions (e.g., Freund's adjuvant); saponin formulations; virosomes and viral-like particles; bacterial and microbial derivatives; immunostimulatory oligonucleotides; ADP-ribosylating toxins and detoxified derivatives; alum; BCG; mineral-containing compositions (e.g., mineral salts, such as aluminium salts and calcium salts, hydroxides, phosphates, sulfates, etc.); bioadhesives and/or mucoadhesives; microparticles; liposomes; polyoxyethylene ether and polyoxyethylene ester formulations; polyphosphazene; muramyl peptides; imidazoquinolone compounds; and surface active substances (e.g. lysolecithin, pluronic polyols, polyanions, peptides, oil emulsions, keyhole limpet hemocyanin, and dinitrophenol).

Adjuvants may also include immunomodulators such as cytokines, interleukins (e.g., IL-1, IL-2, IL-4, IL-5, IL-6, IL-7, IL-12, etc.), interferons (e.g., interferon-.gamma.), macrophage colony stimulating factor, and tumor necrosis factor. Co-stimulatory molecules, including polypeptides of the B7 family, may be administered. Such proteinaceous adjuvants may be provided as the full-length polypeptide or an active fragment thereof, or in the form of DNA, such as plasmid DNA.

Of the main types of antigen-presenting cells (B cell, macrophages and dendritic cells (DCs)), the DC is the most potent and is responsible for initiating all antigen-specific immune responses. Thus, targeting DCs provides the opportunity to enhance the delivery of antigen and antigen responses.

Dendritic cells express a number of cell surface receptors that can mediate the endocytosis of bound antigen. Targeting exogenous antigens to internalizing surface molecules on systemically-distributed antigen presenting cells facilitates uptake of antigens and thus overcomes a major rate-limiting step in immunization and thus in vaccination.

Dendritic cell targeting molecules include monoclonal or polyclonal antibodies or fragments thereof that recognize and bind to epitopes displayed on the surface of dendritic cells. Dendritic cell targeting molecules also include ligands which bind to a cell surface receptor on dendritic cells. One such receptor, the lectin DEC-205, has been used in vitro and in mice to boost both humoral (antibody-based) and cellular (CD8 T cell) responses by 2-4 orders of magnitude (Hawiger, et al., J. Exp. Med., 194(6):769-79 (2001); Bonifaz, et al., J. Exp. Med., 196(12):1627-38 (2002); Bonifaz, et al., J. Exp. Med., 199(6):815-24 (2004)). A variety of other endocytic receptors, including a mannose-specific lectin (mannose receptor) and IgG Fc receptors, have also been targeted in this way with similar enhancement of antigen presentation efficiency. Other suitable receptors which may be targeted include, but are not limited to, DC-SIGN, 33D1, SIGLEC-H, DCIR, CD11c, heat shock protein receptors and scavenger receptors.

Other receptors which may be targeted include the toll-like receptors (TLRs). TLRs recognize and bind to pathogen-associated molecular patterns (PAMPs). PAMPs target the TLR on the surface of the dendritic cell and signals internally, thereby potentially increasing DC antigen uptake, maturation and T-cell stimulatory capacity. PAMPs conjugated to the particle surface or co-encapsulated include unmethylated CpG DNA (bacterial), double-stranded RNA (viral), lipopolysachamide (bacterial), peptidoglycan (bacterial), lipoarabinomannin (bacterial), zymosan (yeast), mycoplasmal lipoproteins such as MALP-2 (bacterial), flagellin (bacterial) poly(inosinic-cytidylic) acid (bacterial), lipoteichoic acid (bacterial) or imidazoquinolines (synthetic).

B. Antibodies

Polyclonal or monoclonal antibodies can be generated using the immunogenic compositions disclosed herein, using standard techniques for immunization and production of single chain recombinant antibodies, polyclonal antibodies or monoclonal antibodies.

As demonstrated in the Examples below, X-isomers of αSyn exhibit enhanced aggregation as compared to native αSyn and are therefore expected to have increased immunogenicity. Antibodies produced using X-αSyn are advantageous because such antibodies are expected to bind to natural αSyn and thus be useful as a therapeutic or for detection of αSyn in subjects or in biological samples.

C. Pharmaceutical Compositions

Pharmaceutical compositions including stabilized conformational isomers of α-synuclein, vaccines containing the same, and antibodies that bind to conformational isomers of α-synuclein are provided. The pharmaceutical compositions may be for administration by oral, parenteral (intramuscular, intraperitoneal, intravenous (IV) or subcutaneous injection), transdermal (either passively or using iontophoresis or electroporation), transmucosal (nasal, vaginal, rectal, or sublingual) routes of administration or using bioerodible inserts and can be formulated in dosage forms appropriate for each route of administration. In one embodiment, the stabilized conformational isomers of α-synuclein, vaccines containing the same, and antibodies that bind to conformational isomers of α-synuclein are administered in an aqueous solution, particularly for parenteral injection. In general, pharmaceutical compositions are provided including effective amounts of stabilized conformational isomers of α-synuclein, vaccines containing the same, antibodies that bind to conformational isomers of α-synuclein, or derivative products, and pharmaceutically acceptable diluents, preservatives, solubilizers, emulsifiers, adjuvants and/or carriers. Such compositions include diluents of various buffer content (e.g., Tris HCl, acetate, phosphate), pH and ionic strength; additives such as detergents and solubilizing agents (e.g., TWEEN® 20, TWEEN® 80, Polysorbate® 80), anti-oxidants (e.g., ascorbic acid, sodium metabisulfite), preservatives (e.g., Thimersol, benzyl alcohol) and bulking substances (e.g., lactose, mannitol); incorporation of the material into particulate preparations of polymeric compounds such as polylactic acid, polyglycolic acid, etc. or into liposomes. Hylauronic acid may also be used. Such compositions may influence the physical state, stability, rate of in vivo release, and rate of in vivo clearance of the proteins and derivatives. See, e.g., Remington's Pharmaceutical Sciences, 18th Ed. (1990, Mack Publishing Co., Easton, Pa. 18042) pages 1435-1712. The compositions may be prepared in liquid form, or may be in dried powder (e.g., lyophilized) form.

Preparations for parenteral administration include sterile aqueous or non-aqueous solutions, suspensions, or emulsions. Examples of non-aqueous solvents or vehicles are propylene glycol, polyethylene glycol, vegetable oils, such as olive oil and corn oil, gelatin, and injectable organic esters such as ethyl oleate. Such dosage forms may also contain adjuvants such as preserving, wetting, emulsifying, and dispersing agents. They may be sterilized by, for example, filtration through a bacteria retaining filter, by incorporating sterilizing agents into the compositions, by irradiating the compositions, or by heating the compositions. They can also be manufactured using sterile water, or some other sterile injectable medium, immediately before use.

### III. Methods of Manufacture

A. Methods for Producing Conformational Isomers of αSyn

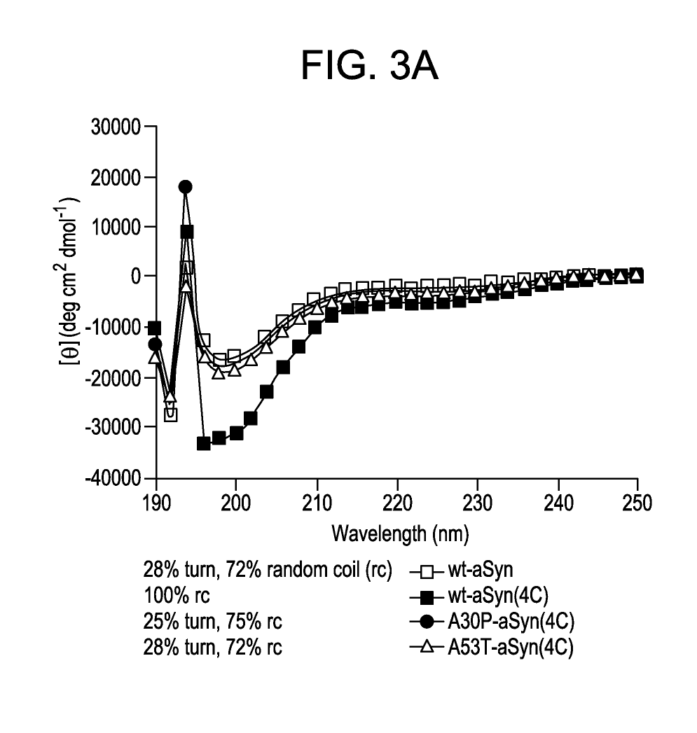

Stabilized conformational isomers of αSyn are generated through the technique of disulfide scrambling. This technique requires proteins that contain at least three cysteines. Native human αSyn does not contain any cysteine residues. The amino acid sequence of wild-type human αSyn is shown in FIG. 3. Therefore, αSyn polypeptides are engineered to contain 3 or more cysteines using methods well known in the art, such as site-directed mutagenesis.

Nucleic acid sequences encoding αSyn polypeptides containing 3 or more cysteines are used to express the polypeptides in prokaryotic or eukaryotic cells as described below in more detail. Following expression and purification, proteins are reduced and denatured in the presence of a reducing agent and a denaturant. After exclusion of the reductant and denaturant, the reduced and denatured proteins are allowed to retold in the presence of a redox buffer. The denaturing and refolding proteins rearrange their disulfide bonds through a series of thiol-disulfide exchange reactions and can assume a number of heterogeneous disulfide isomer intermediates during denaturing and oxidative folding.

Disulfide conformational isomers can be generated in their fully oxidized forms, or may be “trapped” during either denaturing or refolding of the protein by the removal of the thiol catalyst, changing the pH to an acidic pH, or adding thiol trapping reagents, such as iodoacetamine or trifluoracetic acid.

Conformational disulfide isomers of proteins are not interconvertible in the absence of a thiol catalyst or at acidic pH, and thus can be isolated during denaturation or refolding and separated as stable isomers by conventional separation techniques.

This technique produces a pool of scrambled disulfide isomers. Each different species of scrambled disulfide isomer has a unique species-specific protein conformation and each of the species will differ from one another by at least one non-native disulfide. That is, each of the species of protein isomers includes at least one unique pairing of cysteine residues wherein the pairing is not found in the other species or in the native protein. The isomers may differ from one another and from the native protein by at least one, two or more non-native disulfide, e.g., at least two species-specific pairings of cysteine residues.

Individual species of conformational isomers may be further purified and isolated using techniques well known in the art, e.g., precipitation, affinity chromatography, high performance liquid chromatography, fast protein liquid chromatography, two and three-dimensional gel electrophoresis, MALDI, MALDI-TOF, and combinations thereof.

1. Expression of αSyn Polypeptides

Isolated αSyn polypeptides containing 3 or more cysteine residues can be obtained by, for example, chemical synthesis or by recombinant production in a host cell. To recombinantly produce an αSyn polypeptide, a nucleic acid containing a nucleotide sequence encoding an αSyn polypeptide containing 3 or more cysteines can be used to transform, transduce, or transfect a bacterial or eukaryotic host cell (e.g., an insect, yeast, or mammalian cell). Nucleic acids expressing mutants of αSyn containing 3 or more cysteines can be inserted into vectors for expression in cells. As used herein, a “vector” is a replicon, such as a plasmid, phage, or cosmid, into which another DNA segment may be inserted so as to bring about the replication of the inserted segment. Vectors can be expression vectors. An “expression vector” is a vector that includes one or more expression control sequences, and an “expression control sequence” is a DNA sequence that controls and regulates the transcription and/or translation of another DNA sequence.

Nucleic acids in vectors can be operably linked to one or more expression control sequences. As used herein, “operably linked” means incorporated into a genetic construct so that expression control sequences effectively control expression of a coding sequence of interest. Examples of expression control sequences include promoters, enhancers, and transcription terminating regions. A promoter is an expression control sequence composed of a region of a DNA molecule, typically within 100 nucleotides upstream of the point at which transcription starts (generally near the initiation site for RNA polymerase II). To bring a coding sequence under the control of a promoter, it is necessary to position the translation initiation site of the translational reading frame of the polypeptide between one and about fifty nucleotides downstream of the promoter. Enhancers provide expression specificity in terms of time, location, and level. Unlike promoters, enhancers can function when located at various distances from the transcription site. An enhancer also can be located downstream from the transcription initiation site. A coding sequence is “operably linked” and “under the control” of expression control sequences in a cell when RNA polymerase is able to transcribe the coding sequence into mRNA, which then can be translated into the protein encoded by the coding sequence.

Suitable expression vectors include, without limitation, plasmids and viral vectors derived from, for example, bacteriophage, baculoviruses, tobacco mosaic virus, herpes viruses, cytomegalo virus, retroviruses, vaccinia viruses, adenoviruses, and adeno-associated viruses. Numerous vectors and expression systems are commercially available from such corporations as Novagen (Madison, Wis.), Clontech (Palo Alto, Calif.), Stratagene (La Jolla, Calif.), and Invitrogen Life Technologies (Carlsbad, Calif.).

An expression vector can include a tag sequence. Tag sequences, are typically expressed as a fusion with the encoded polypeptide. Such tags can be inserted anywhere within the polypeptide including at either the carboxyl or amino terminus. Examples of useful tags include, but are not limited to, green fluorescent protein (GFP), glutathione S-transferase (GST), polyhistidine, c-myc, hemagglutinin, Flag™ tag (Kodak, New Haven, Conn.), maltose E binding protein and protein A.

Vectors containing nucleic acids to be expressed can be transferred into host cells. The term “host cell” is intended to include prokaryotic and eukaryotic cells into which a recombinant expression vector can be introduced. As used herein, “transformed” and “transfected” encompass the introduction of a nucleic acid molecule (e.g., a vector) into a cell by one of a number of techniques. Although not limited to a particular technique, a number of these techniques are well established within the art. Prokaryotic cells can be transformed with nucleic acids by, for example, electroporation or calcium chloride mediated transformation. Nucleic acids can be transfected into mammalian cells by techniques including, for example, calcium phosphate co-precipitation, DEAE-dextran-mediated transfection, lipofection, electroporation, or microinjection. Host cells (e.g., a prokaryotic cell or a eukaryotic cell such as a CHO cell) can be used to, for example, produce the variant αSyn polypeptides described herein. In some embodiments, a host cell (e.g., an antigen presenting cell) can be used to express the variant αSyn polypeptides disclosed herein for presentation to a T cell.

Useful prokaryotic and eukaryotic systems for expressing and producing polypeptides are well know in the art include, for example, Escherichia coli strains such as BL-21, and cultured mammalian cells such as CHO cells.

In eukaryotic host cells, a number of viral-based expression systems can be utilized to express αSyn polypeptides containing 3 or more cysteines. Viral based expression systems are well known in the art and include, but are not limited to, baculoviral, SV40, retroviral, or vaccinia based viral vectors.

Mammalian cell lines that stably express αSyn polypeptides containing 3 or more cysteines can be produced using expression vectors with appropriate control elements and a selectable marker. For example, the eukaryotic expression vectors pCR3.1 (Invitrogen Life Technologies) and p91023(B) (see Wong et al. (1985) Science 228:810-815) are suitable for expression of variant costimulatory polypeptides in, for example, Chinese hamster ovary (CHO) cells, COS-1 cells, human embryonic kidney 293 cells, NIH3T3 cells, BHK21 cells, MDCK cells, and human vascular endothelial cells (HUVEC). Following introduction of an expression vector by electroporation, lipofection, calcium phosphate, or calcium chloride co-precipitation, DEAE dextran, or other suitable transfection method, stable cell lines can be selected (e.g., by antibiotic resistance to G418, kanamycin, or hygromycin). The transfected cells can be cultured such that the polypeptide of interest is expressed, and the polypeptide can be recovered from, for example, the cell culture supernatant or from lysed cells. Alternatively, αSyn polypeptides containing 3 or more cysteines can be produced by (a) ligating amplified sequences into a mammalian expression vector such as pcDNA3 (Invitrogen Life Technologies), and (b) transcribing and translating in vitro using wheat germ extract or rabbit reticulocyte lysate.

αSyn polypeptides containing 3 or more cysteines can be isolated using, for example, chromatographic methods such as DEAE ion exchange, gel filtration, and hydroxylapatite chromatography. For example, a costimulatory polypeptide in a cell culture supernatant or a cytoplasmic extract can be isolated using a protein G column. In some embodiments, variant costimulatory polypeptides can be “engineered” to contain an amino acid sequence that allows the polypeptides to be captured onto an affinity matrix. For example, a tag such as c-myc, hemagglutinin, polyhistidine, or Flag™ (Kodak) can be used to aid polypeptide purification. Such tags can be inserted anywhere within the polypeptide, including at either the carboxyl or amino terminus. Other fusions that can be useful include enzymes that aid in the detection of the polypeptide, such as alkaline phosphatase. Immunoaffinity chromatography also can be used to purify costimulatory polypeptides.

2. Denaturation and Reduction

Various protein denaturing agents and conditions include, but are not limited to, one or more concentrations of urea, guanidinium chloride, guanidinium isothiocyanate, organic solvents, elevated temperature, extreme pH, surfactants and detergents, mechanical forces such as shaking, shearing, ultrasound, radiation and pressure and mixtures and combinations thereof.

Examples of reducing agents include, but are Dot limited to, 2-mercaptoethanol, reduced glutathione, cysteine, dithiothreitol, and thiol-containing chemicals.

The applied denaturing and/or reducing conditions can be titrated according to time, concentration or other relevant variables to increase the diversity of stabilized conformational isomers obtained.

3. Oxidation

αSyn polypeptides containing 3 or more cysteines can be allowed to refold in standard buffer conditions without redox agents or with the addition of redox agents with oxygen from air being responsible for disulfide oxidation. In one embodiment the redox agent is CuSO₄. Copper ions are known to catalyze air-oxidation of thiol groups to disulfide bonds. The Examples below demonstrate that inclusion of CuSO₄ in the oxidation buffer greatly enhances oxidation of X-αSyn polypeptides and, as a result, greatly enhances the diversity of conformational isomers of the refolded polypeptides.

B. Methods for Producing Antibodies

The basic antibody structural unit is known to comprise a tetramer of subunits. Each tetramer is composed of two identical pairs of polypeptide chains, each pair having one “light” (about 25kDa) and one “heavy” chain (about 50-70 kDa). The amino-terminal portion of each chain includes a variable region of about 100 to 10 or more amino acids primarily responsible for antigen recognition. The carboxy-terminal portion of each chain defines a constant region primarily responsible for effector function.

Light chains are classified as either kappa or lambda. Heavy chains are classified as gamma, mu, alpha, delta; or epsilon, and define the antibody's isotype as IgG, IgM, IgA, IgD and IgE, respectively. Within light and heavy chains, the variable and constant regions are joined by a “J” region of about 12 or more amino acids, with the heavy chain also including a “D” region of about 10 more amino acids. (See generally, Fundamental Immunology, Paul, W., ed., 2nd ed. Raven Press, N.Y., 1989, Ch. 7).

The variable regions of each light/heavy chain pair form the antibody binding site. Thus, an intact antibody has two binding sites. Except in bifunctional or bispecific antibodies, the two binding sites are the same. The chains all exhibit the same general structure of relatively conserved framework regions (FR) joined by three hypervariable regions, also called complementarity determining regions or CDRs. The CDRs from the two chains of each pair are aligned by the framework regions, enabling binding to a specific epitope. From N-terminal to C-terminal, both light and heavy chains comprise the domains FR1, CDR1, FR2, CDR2, FR3, CDR3 and FR4.

1. Production of Polyclonal Antibodies

Polyclonal antibodies are obtained as sera from immunized animals such as rabbits, goats, rodents, etc. and may be used directly without further treatment or may be subjected to conventional enrichment or purification methods such as ammonium sulfate precipitation, ion exchange chromatography, and affinity chromatography.

2. Production of Monoclonal Antibodies

Monoclonal antibodies may be produced using conventional hybridoma technology, such as the procedures introduced by Kohler and Milstein Nature, 256:495-97 (1975)), and modifications thereof (see above references). An animal, preferably a mouse is primed by immunization with an immunogen as above to elicit the desired antibody response in the primed animal. B lymphocytes from the lymph nodes, spleens or peripheral blood of a primed, animal are fused with myeloma cells, generally in the presence of a fusion promoting agent such as polyethylene glycol (PEG). Any of a number of murine myeloma cell lines are available for such use: the P3-NS1/1-Ag4-1, P3-x63-k0Ag8.653, Sp2/0-Ag14, or HL1-653 myeloma lines (available from the ATCC, Rockville, Md.). Subsequent steps include growth in selective medium so that unfused parental myeloma cells and donor lymphocyte cells eventually die while only the hybridoma cells survive. These are cloned and grown and their supernatants screened for the presence of antibody of the desired specificity, e.g. by immunoassay techniques using B7-DC fusion proteins. Positive clones are subcloned, e.g., by limiting dilution, and the monoclonal antibodies are isolated.

Hybridomas produced according to these methods can be propagated in vitro or in vivo (in ascites fluid) using techniques known in the art (see generally Fink et al., Prog. Clin. Pathol, 9:121-33 (1984)). Generally, the individual cell line is propagated in culture and the culture medium containing high concentrations of a single monoclonal antibody can be harvested by decantation, filtration, or centrifugation.

a. Production of Chimeric and Humanized Monoclonal Antibodies

Chimeric and humanized antibodies have the same or similar binding specificity and affinity as a mouse or other nonhuman antibody that provides the starting material for construction of a chimeric or humanized antibody. Chimeric antibodies are antibodies whose light and heavy chain genes have been constructed, typically by genetic engineering, from immunoglobulin gene segments belonging to different species. For example, the variable (V) segments of the genes from a mouse monoclonal antibody may be joined to human constant (C) segments, such as IgG1 and IgG4. Human isotype IgG1 is preferred. In some methods, the isotype of the antibody is human IgG1. IgM antibodies can also be used in some methods. A typical chimeric antibody is thus a hybrid protein consisting of the V or antigen-binding domain from a mouse antibody and the C or effector domain from a human antibody.

Humanized antibodies have variable region framework residues substantially from a human antibody (termed an acceptor antibody) and complementarity determining regions substantially from a mouse-antibody, (referred to as the donor immunoglobulin). See, Queen et al., Proc. Natl. Acad. Sci. USA 86:10029-10033 (1989), WO 90/07861, U.S. Pat. No. 5,693,762, U.S. Pat. No. 5,693,761, U.S. Pat. No. 5,585,089, U.S. Pat. No. 5,530,101, and Winter, U.S. Pat. No. 5,225,539). The constant region(s), if present, are also substantially or entirely from a human immunoglobulin. The human variable domains are usually chosen from human antibodies whose framework sequences exhibit a high degree of sequence identity with the murine variable region domains from which the CDRs were derived. The heavy and light chain variable region framework residues can be derived from the same or different human antibody sequences. The human antibody sequences can be the sequences of naturally occurring human antibodies or can be consensus sequences of several human antibodies. Certain amino acids from the human variable region framework residues are selected for substitution based on their possible influence on CDR conformation and/or binding to antigen. Investigation of such possible influences is by modeling, examination of the characteristics of the amino acids at particular locations, or empirical observation of the effects of substitution or mutagenesis of particular amino acids.

For example, when an amino acid differs between a murine variable region framework residue and a selected human variable region framework residue, the human framework amino acid should usually be substituted by the equivalent framework amino acid from the mouse antibody when it is reasonably expected that the amino acid:

(1) noncovalently binds antigen directly,

(2) is adjacent to a CDR region,

(3) otherwise interacts with a CDR region (e.g. is within about 6 A of a CDR region), or

(4) participates in the VL-VH interface.

Other candidates for substitution are acceptor human framework amino acids that are unusual for a human immunoglobulin at that position. These amino acids can be substituted with amino acids from the equivalent position of the mouse donor antibody or from the equivalent positions of more typical human immunoglobulins. Other candidates for substitution are acceptor human framework amino acids that are unusual for a human immunoglobulin at that position. The variable region frameworks of humanized immunoglobulins usually show at least 85% sequence identity to a human variable region framework sequence or consensus of such sequences.

b. Production of Human Monoclonal Antibodies

Human antibodies against stabilized conformational isomers of α-synuclein are provided by a variety of techniques described below. Some human antibodies are selected by competitive binding experiments, or otherwise, to have the same epitope specificity as a particular mouse antibody. Human antibodies preferably have isotype specificity human IgG1.

One method for producing human monoclonal antibodies is the trioma methodology. The basic approach and an exemplary cell fusion partner, SPAZ-4, for use in this approach have been described by Oestberg et al., Hybridoma 2:361-367 (1983); Oestberg, U.S. Pat. No. 4,634,664; and Engleman et al., U.S. Pat. No. 4,634,666). The antibody-producing cell lines obtained by this method are called triomas, because they are descended from three cells-two human and one mouse. Initially, a mouse myeloma line is fused with a human B-lymphocyte to obtain a non-antibody-producing xenogeneic hybrid cell, such as the SPAZ-4 cell line. The xenogeneic cell is then fused with an immunized human B-lymphocyte to obtain an antibody-producing trioma cell line. Triomas have been found to produce antibody more stably than ordinary hybridomas made from human cells.

The immunized B-lymphocytes are obtained from the blood, spleen, lymph nodes or bone marrow of a human donor. If antibodies against a specific antigen or epitope are desired, it is preferable to use that antigen or epitope thereof for immunization. Immunization can be either in vivo or in vitro. For in vivo immunization, B cells are typically isolated from a human immunized with stabilized conformational isomers of αSyn. In some methods, B cells are isolated from the same patient who is ultimately to be administered antibody therapy. For in vitro immunization, B-lymphocytes are typically exposed to antigen for a period of 7-14 days in a media such as RPMI-1640 supplemented with 10% human plasma.

The immunized B-lymphocytes are fused to a xenogeneic hybrid cell such as SPAZ-4 by well known methods. For example, the cells are treated with 40-50% polyethylene glycol of MW 1000-4000, at about 37° C., for about 5-10 min. Cells are separated from the fusion mixture and propagated in media selective for the desired hybrids (e.g., HAT or AH). Clones secreting antibodies having the required binding specificity are identified by assaying the trioma culture medium for the ability to bind to αSyn. Triomas producing human antibodies having the desired specificity are subcloned by the limiting dilution technique and grown in vitro in culture medium. The trioma cell lines obtained are then tested for the ability to bind αSyn.

Although triomas are genetically stable they do not produce antibodies at very high levels. Expression levels can be increased by cloning antibody genes from the trioma into one or more expression vectors, and transforming the vector into standard mammalian, bacterial or yeast cell lines.

Human antibodies against αSyn can also be produced from non-human transgenic mammals having transgenes encoding at least a segment of the human immunoglobulin locus. Usually, the endogenous immunoglobulin locus of such transgenic mammals is functionally inactivated. Preferably, the segment of the human immunoglobulin locus includes unrearranged sequences of heavy and light chain components. Both inactivation of endogenous immunoglobulin genes and introduction of exogenous immunoglobulin genes can be achieved by targeted homologous recombination, or by introduction of YAC chromosomes. The transgenic mammals resulting from this process are capable of functionally rearranging the immunoglobulin component sequences, and expressing a repertoire of antibodies of various isotypes encoded by human immunoglobulin genes, without expressing endogenous immunoglobulin genes. The production and properties of mammals having these properties are described in detail by, e.g., Lonberg et al., WO93/1222, U.S. Pat. No. 5,877,397, U.S. Pat. No. 5,874,299, U.S. Pat. No. 5,814,318, U.S. Pat. No. 5,789,650, U.S. Pat. No. 5,770,429, U.S. Pat. No. 5,661,016, U.S. Pat. No. 5,633,425, U.S. Pat. No. 5,625,126, U.S. Pat. No. 5,569,825, U.S. Pat. No. 5,545,806, Nature 148, 1547-1553 (1994), Nature Biotechnology 14, 826 (1996), Kucherlapati, WO 91/10741. Transgenic mice are particularly suitable. Anti-αSyn antibodies are obtained by immunizing a transgenic nonhuman mammal with stabilized conformational isomers of αSyn. Monoclonal antibodies are prepared by, e.g., fusing B-cells from such mammals to suitable myeloma cell lines using conventional Kohler-Milstein technology. Human polyclonal antibodies can also be provided in the form of serum from humans immunized with an immunogenic agent. Optionally, such polyclonal antibodies can be concentrated by affinity purification using αSyn as an affinity reagent.

A further approach for obtaining human anti-αSyn antibodies is to screen a DNA library from human B cells according to the general protocol outlined by Huse et al., Science 246:1275-1281 (1989). As described for trioma methodology, such B cells can be obtained from a human immunized with stabilized conformational isomers of αSyn. Optionally, such B cells are obtained from a patient who is ultimately to receive antibody treatment. Antibodies binding to αSyn or a fragment thereof are selected. Sequences encoding such antibodies (or binding fragments) are then cloned and amplified. The protocol described by Huse is rendered more efficient in combination with phage-display technology. See, e.g., Dower et al., WO 91/17271 and McCafferty et al., WO 92/01047, U.S. Pat. No. 5,877,218, U.S. Pat. No. 5,871,907, U.S. Pat. No. 5,858,657, U.S. Pat. No. 5,837,242, U.S. Pat. No. 5,733,743 and U.S. Pat. No. 5,565,332). In these methods, libraries of phage are produced in which members display different antibodies on their outer surfaces. Antibodies are usually displayed as Fv or Fab fragments. Phage displaying antibodies with a desired specificity are selected by affinity enrichment to an αSyn peptide or fragment thereof.

In a variation of the phage-display method, human antibodies having the binding specificity of a selected murine antibody can be produced (Winter, WO 92/20791). In this method, either the heavy or light chain variable region of the selected murine antibody is used as a starting material. If, for example, a light chain variable region is selected as the starting material, a phage library is constructed in which members display the same light chain variable region (i.e., the murine starting material) and a different heavy chain variable region. The heavy chain variable regions are obtained from a library of rearranged human heavy chain variable regions. A phage showing strong specific binding for αSyn (e.g., at least 10⁸ and preferably at least 10⁹M⁻¹) is selected. The human heavy chain variable region from this phage then serves as a starting material for constructing a further phage library. In this library, each phage displays the same heavy chain variable region (i.e., the region identified from the first display library) and a different light chain variable region. The light chain variable regions are obtained from a library of rearranged human variable light chain regions. Again, phage showing strong specific binding for αSyn are selected. These phage display the variable regions of completely human anti-αSyn antibodies. These antibodies usually have the same or similar epitope specificity as the murine starting material.

The heavy and light chain variable regions of chimeric, humanized, or human antibodies can be linked to at least a portion of a human constant region. The choice of constant region depends, in part, whether antibody-dependent complement and/or cellular mediated toxicity is desired. For example, isotopes IgG1 and IgG3 have complement activity and isotypes IgG2 and IgG4 do not. Choice of isotype can also affect passage of antibody into the brain. Human isotype IgG1 is preferred. Light chain constant regions can be lambda or kappa. Antibodies can be expressed as tetramers containing two light and two heavy chains, as separate heavy chains, light chains, as Fab, Fab′F(ab′)2, and Fv, or as single chain antibodies in which heavy and light chain variable domains are linked through a spacer.

3. Expression of Recombinant Antibodies

Chimeric, humanized and human antibodies are typically produced by recombinant expression. Recombinant polynucleotide constructs typically include an expression control sequence operably linked to the coding sequences of antibody chains, including naturally associated or heterologous promoter regions. Preferably, the expression control sequences are eukaryotic promoter systems in vectors capable of transforming or transfecting eukaryotic host cells. Once the vector has been incorporated into the appropriate host, the host is maintained under conditions suitable for high level expression of the nucleotide sequences, and the collection and purification of the crossreacting antibodies. These expression vectors are typically replicable in the host organisms either as episomes or as an integral part of the host chromosomal DNA. Commonly, expression vectors contain selection markers, e.g., ampicillin-resistance or hygromycin-resistance, to permit detection of those cells transformed with the desired DNA sequences.

E. coli is one prokaryotic host particularly useful for cloning the DNA sequences of the present invention. Microbes such as yeast are also useful for expression. Saccharomyces is a preferred yeast host, with suitable vectors having expression control sequences, an origin of replication, and termination sequences as desired. Typical promoters include 3-phosphoglycerate kinase and other glycolytic enzymes. Inducible yeast promoters include, among others, promoters from alcohol dehydrogenase, isocytochrome C, and enzymes responsible for maltose and galactose utilization.

Mammalian cells are a preferred host for expressing nucleotide segments encoding immunoglobulins or fragments thereof (Winnacker, From Genes to Clones, VCH Publishers, NY, 1987). A number of suitable host cell lines capable of secreting intact heterologous proteins have been developed in the art, and include CHO cell lines, various COS cell lines, HeLa cells, L cells, human embryonic kidney cell, and myeloma cell lines. Preferably, the cells are non-human. Expression vectors for these cells can include expression control sequences, such as an origin of replication, a promoter an enhancer (Queen et al., Immunol. Rev. 89:49 (1986)), and necessary processing information sites, such as ribosome binding sites, RNA splice sites, polyadenylation sites, and transcriptional terminator sequences. Preferred expression control sequences are promoters derived from endogenous genes including cytomegalovirus, SV40, adenovirus, bovine papillomavirus (Co et al., J. Immunol. 148:1149 (1992).

Alternatively, antibody coding sequences can be incorporated in transgenes for introduction into the genome of a transgenic animal and subsequent expression in the milk of the transgenic animal (see, e.g., U.S. Pat. No. 5,741,957, U.S. Pat. No. 5,304,489, U.S. Pat. No. 5,849,992). Suitable transgenes include coding sequences for light and/or heavy chains in operable linkage with a promoter and enhancer from a mammary gland specific gene, such as casein or beta lactoglobulin.

The vectors containing the DNA segments of interest can be transferred into the host cell by well-known methods, depending on the type of cellular host. For example, calcium chloride transfection is commonly utilized for prokaryotic cells, whereas calcium phosphate treatment, electroporation, lipofection, biolistics or viral-based transfection can be used for other cellular hosts. Other methods used to transform mammalian cells include the use of polybrene, protoplast fusion, liposomes, electroporation, and microinjection (see generally, Sambrook et al., supra). For production of transgenic animals, transgenes can be microinjected into fertilized oocytes, or can be incorporated into the genome of embryonic stem cells, and the nuclei of such cells transferred into enucleated oocytes.

Once expressed, antibodies can be purified according to standard procedures of the art, including HPLC purification, column chromatography, gel electrophoresis and the like (see generally, Scopes, Protein Purification (Springer-Verlag, NY, 1982)).

Polypeptide immunogens disclosed herein can also be linked to a suitable carrier molecule to form a conjugate which helps elicit an immune response. Suitable carriers include serum albumins, keyhole limpet hemocyanin, immunoglobulin molecules, thyroglobulin, ovalbumin, tetanus toxoid, or a toxoid from other pathogenic bacteria, such as diphtheria, E. coli, cholera, or H. pylori, or an attenuated toxin derivative. T cell epitopes are also suitable carrier molecules. Some conjugates can be formed by linking agents of the invention to an immunostimulatory polymer molecule (e.g., tripalmitoyl-5-glycerine cysteine (Pam₃Cys), mannan (a manose polymer), or glucan (a beta 1.fwdarw.2 polymer)), cytokines (e.g., IL-1, IL-1 alpha and beta peptides, IL-2, gamma-INF, IL-10, GM-CSF), and chemokines (e.g., MIP1alpha and beta, and RANTES). Immunogenic agents can also be linked to peptides that enhance transport across tissues, as described in O'Mahony, WO 97/17613 and WO 97/17614. Immunogens may be linked to the carriers with or with out spacers amino acids (e.g., gly-gly).

Some conjugates can be formed by linking agents to at least one T cell epitope. Some T cell epitopes are promiscuous while other T cell epitopes are universal. Promiscuous T cell epitopes are capable of enhancing the induction of T cell immunity in a wide variety of subjects displaying various HLA types. In contrast to promiscuous T cell epitopes, universal T cell epitopes are capable of enhancing the induction of T cell immunity in a large percentage, e.g., at least 75%, of subjects displaying various HLA molecules encoded by different HLA-DR alleles.

A large number of naturally occurring T-cell epitopes exist, such as, tetanus toxoid (e.g., the P2 and P30 epitopes), Hepatitis B surface antigen, pertussis, toxoid, measles virus F protein, Chlamydia trachomitis major outer membrane protein, diphtheria toxoid, Plasmodium falciparum circumsporozite T, Plasmodium falciparum CS antigen, Schistosoma mansoni triose phosphate isomersae, Escherichia coli TraT, and Influenza virus hemaglutinin (HA). The immunogenic peptides of the invention can also be conjugated to the T-cell epitopes described in Sinigaglia, et al., Nature, 336:778-780 (1988); Chicz, et al., J. Exp. Med., 178:27-47 (1993); Hammer, et al., Cell, 74:197-203 (1993); Falk, et al., Immunogenetics, 39:230-242 (1994); and, Southwood, et al., J. Immunology, 160:3363-3373 (1998).

Alternatively, the conjugates can be formed by linking agents to at least one artificial T-cell epitope capable of binding a large proportion of MHC Class II molecules, such as the pan DR epitope (“PADRE”). PADRE is described in U.S. Pat. No. 5,736,142, WO 95/07707, and Alexander J et al., Immunity, 1:751-761 (1994). A preferred PADRE peptide is AKXVAAWTLKAAA, wherein X is preferably cyclohexylalanine, tyrosine or phenylalanine, with cyclohexylalanine being most preferred.

Immunogenic agents can be linked to carriers by chemical crosslinking. Techniques for linking an immunogen to a carrier include the formation of disulfide linkages using N-succinimidyl-3-(2-pyridyl-thio)propionate (SPDP) and succinimidyl 4-(N-maleimidomethyl)cyclohexane-1-carboxylate (SMCC) (if the peptide lacks a sulfhydryl group, this can be provided by addition of a cysteine residue). These reagents create a disulfide linkage between themselves and peptide cysteine resides on one protein and an amide linkage through the epsilon-amino on a lysine, or other free amino group in other amino acids. A variety of such disulfide/amide-forming agents are described by Immun. Rev. 62, 185 (1982). Other bifunctional coupling agents form a thioether rather than a disulfide linkage. Many of these thio-ether-forming agents are commercially available and include reactive esters of 6-maleimidocaproic acid, 2-bromoacetic acid, and 2-iodoacetic acid, 4-(N-maleimido-methyl)cyclohexane-1-carboxylic acid. The carboxyl groups can be activated by combining them with succinimide or 1-hydroxyl-2-nitro-4-sulfonic acid, sodium salt.

Immunogenicity can be improved through the addition of spacer residues (e.g., Gly-Gly) between the Tₕ epitope and the peptide immunogen. In addition to physically separating the Tₕ epitope from the B cell epitope (i.e., the peptide immunogen), the glycine residues can disrupt any artificial secondary structures created by the joining of the Tₕ epitope with the peptide immunogen, and thereby eliminate interference between the T and/or B cell responses. The conformational separation between the helper epitope and the antibody eliciting domain thus permits more efficient interactions between the presented immunogen and the appropriate Tₕ and B cells.

To enhance the induction of T cell immunity in a large percentage of subjects displaying various HLA types to an agent of the present invention, a mixture of conjugates with different Tₕ cell epitopes can be prepared. The mixture may contain a mixture of at least two conjugates with different Tₕ cell epitopes, a mixture of at least three conjugates with different Tₕ cell epitopes, or a mixture of at least four conjugates with different Tₕ cell epitopes. The mixture may be administered with an adjuvant.

Immunogenic peptides can also be expressed as fusion proteins with carriers (i.e., heterologous peptides). The immunogenic peptide can be linked at its amino terminus, its carboxyl terminus, or both to a carrier. Optionally, multiple repeats of the immunogenic peptide can be present in the fusion protein. Optionally, an immunogenic peptide can be linked to multiple copies of a heterologous peptide, for example, at both the N and C termini of the peptide. Some carrier peptides serve to induce a helper T-cell response against the carrier peptide. The induced helper T-cells in turn induce a B-cell response against the immunogenic peptide linked to the carrier peptide.

### IV. Methods of Use

A. Diseases and Disorders to be Diagnosed, Prevented or Treated

The vaccines and antibodies are generally useful for prophylactic or therapeutic treatment of subjects having or being predisposed to symptoms of synucleinopathies, including Parkinson's disease (PD), and Lewy body diseases, including diffuse dementia with Lewy bodies (DLB), Lewy body disease (DLBD), Lewy body variant of Alzheimer's disease (LBVAD), combined PD and Alzheimer's disease (AD), multiple systems atrophy, and neurodegeneration with brain iron accumulation type-1 (NBIA-1).

The vaccines are also useful for the production of antibodies which can be used for prevention or treatment of symptoms of synucleinopathies in subjects having or being predisposed to development of synucleinopathies. The antibodies are also useful for diagnosis of synucleinopathies in individuals.

B. Vaccines

The effectiveness of an antigen in inducing an immune response against a particular antigen can depend upon several factors, many of which are not well understood. Most previously available methods for increasing the effectiveness of antigens are dependent upon understanding the molecular basis for these factors. However, immunization with a pool of non-native protein isomers are effective even where the molecular bases are unknown. Assumptions or calculations of potential antigenicity of αSyn compositions are not required because dozens, hundreds or even thousands of potential conformational variants of αSyn may be tested alone or as pools of antigens.

The rapid development of many multiples of antigenic determinants for αSyn is accomplished through disulfide scrambling to create one or more pools of potential antigens. Diversity of αSyn conformational isomers is achieved, as described above, by varying the number and location of cysteines within the amino acid sequence, as well as by varying the denaturing, reducing, and/or refolding conditions. The immunogenic compositions described herein provide many potential antigenic epitopes for both B-cell and T-cell activation, and immunogenic combinations thereof, for α-synuclein.

Pools of αSyn conformational isomers can be screened to determine efficacy. That pool of αSyn conformational isomers that is most protective can be selected from these pools by in vivo challenge models. Individual non-native isomers of α-synuclein that are antigenic in an individual patient can also be identified, providing thereby a potential customized, patient-specific antigen.

The vaccines disclosed herein can be used for prophylactic and therapeutic vaccination. In prophylactic applications, vaccines including stabilized conformational isomers of α-synuclein, and antibodies thereto, are provided in amounts and frequencies of administration sufficient to eliminate or reduce the risk or delay the outset of synucleinopathies, including physiological, biochemical, histologic and/or behavioral symptoms of the disease, its complications and intermediate pathological phenotypes presenting during development of the disease. In therapeutic applications, the compositions disclosed herein are administered to a patient suspected of, or already suffering from such a disease in a regime including an amount and frequency of administration of the composition sufficient to result in reduction of intracellular levels of aggregated α-synuclein. In other embodiments, vaccines and antibodies disclosed herein are administered in effective amounts and frequencies to treat, at least partially, the symptoms of the disease (physiological, biochemical, histologic and/or behavioral), including its complications and intermediate pathological phenotypes in development of the disease. An amount adequate to accomplish therapeutic or prophylactic treatment is defined as a therapeutically- or prophylactically-effective amount. For both prophylactic and therapeutic administration, vaccines are usually administered in several dosages until a sufficient immune response has been achieved. Typically, the immune response is monitored and repeated dosages are given if the immune response starts to wane.

For vaccines including conformational isomers of α-synuclein, and antibodies that bind to conformational isomers of α-synuclein, as further studies are conducted, information will emerge regarding appropriate dosage levels for treatment or prevention of various conditions in various patients, and the ordinary skilled worker, considering the therapeutic context, age, and general health of the recipient, will be able to ascertain proper dosing. The selected dosage depends upon the desired therapeutic effect, on the route of administration, and on the duration of the treatment desired. Generally, for intravenous injection or infusion, dosages may be lower than other routes of administration.

C. Diagnosis and Treatment of α-Synucleinopathies Using Antibodies

One embodiment provides methods for diagnosing α-synucleinopathies in a host using antibodies that bind to αSyn. Antibodies can be applied to biological samples derived from the host to quantify the amount of αSyn present in the sample. The antibodies may be labeled radioactively, fluorescently or by attachment of an enzyme that catalyzes a reaction to produce a detectable signal. Alternatively, a secondary antibody that recognizes and binds to the antibodies disclosed herein can be labeled.

The antibodies can also be used for in vivo imaging of Lewy Bodies a patient. The antibodies can be administered to the patient by intravenous injection, or by intracranial injection.

D. Dosages

1. Vaccines

The amount of immunogen administered depends on whether adjuvant is also administered, with higher dosages being required in the absence of adjuvant. The amount of an immunogen for administration sometimes varies from 1-500 μg per patient and more usually from 5-500 μg per injection for human administration. Occasionally, a higher dose of 1-2 mg per injection is used. Typically about 10, 20, 50 or 100 μg is used for each human injection. The mass of immunogen also depends on the mass ratio of immunogenic epitope within the immunogen to the mass of immunogen as a whole. Typically, 10⁻³ to 10⁻⁵ micromoles of immunogenic epitope are used per microgram of immunogen. The timing of injections can vary significantly from once a day, to once a year, to once a decade. On any given day that a dosage of immunogen is given, the dosage is greater than 1 μg/patient and usually greater than 10 μg/patient if adjuvant is also administered, and greater than 10 μg/patient and usually greater than 100 μg/patient in the absence of adjuvant. A typical regimen consists of an immunization followed by booster injections at time intervals, such as 6 week intervals. Another regimen consists of an immunization followed by booster injections 1, 2 and 12 months later. Another regimen entails an injection every two months for life. Alternatively, booster injections can be on an irregular basis as indicated by monitoring of immune response.

2. Therapeutic Antibody Compositions

For antibody administration, the dosage ranges from about 0.0001 to 100 mg/kg, and more usually 0.01 to 5 mg/kg, of the host body weight. For example dosages can be 1 mg/kg body weight or 10 mg/kg body weight or within the range of 1-10 mg/kg or, in other words, 70 mgs or 700 mgs or within the range of 70-700 mgs, respectively, for a 70 kg patient. An exemplary treatment regime entails administration once per every two weeks or once a month or once every 3 to 6 months. In some methods, two or more monoclonal antibodies with different binding specificities are administered simultaneously, in which case the dosage of each antibody administered falls within the ranges indicated. Antibody is usually administered on multiple occasions. Intervals between single dosages can be weekly, monthly or yearly. Intervals can also be irregular as indicated by measuring blood levels of antibody to αSyn in the patient. In some methods, dosage is adjusted to achieve a plasma antibody concentration of 1-1000 μg/ml and in some methods 25-300 μg/ml. Alternatively, antibody can be administered as a sustained release formulation, in which case less frequent administration is required. Dosage and frequency vary depending on the half-life of the antibody in the patient. In general, human antibodies show the longest half life, followed by humanized antibodies, chimeric antibodies, and nonhuman antibodies. The dosage and frequency of administration can vary depending on whether the treatment is prophylactic or therapeutic. In prophylactic applications, a relatively low dosage is administered at relatively infrequent intervals over a long period of time. Some patients continue to receive treatment for the rest of their lives. In therapeutic applications, a relatively high dosage at relatively short intervals is sometimes required until progression of the disease is reduced or terminated, and preferably until the patient shows partial or complete amelioration of symptoms of disease. Thereafter, the patent can be administered a prophylactic regime.

### EXAMPLES

The present invention may be further understood by reference to the following non-limiting examples.

### Example 1

### Rapid Purification of α-Synuclein Proteins

Materials and Methods:

Nomenclatures of Mutants and Isomers of α-Synuclein:

To facilitate the description of various isomers and mutants of α-synuclein throughout the examples, the following measures are taken; (a) α-Synuclein is abbreviated as αSyn; (b) The naturally occurring mutations are added as a prefix. For instance, wt-αSyn, A30P-αSyn and A53T-αSyn represent wild-type, A30P and A53T αSyn respectively; (c) Native αSyn (wt-αSyn) does not comprise a disulfide bond. In order to generate conformational isomers of αSyn, a recombinant αSyn with 4 Ala/Ser [symbol] Cys mutations (at positions 9, 42, 69 and 89) was expressed and purified in reduced form as αSyn(4C). The introduced Ala/Ser [symbol] Cys mutations are attached as a suffix. For example, wt-αSyn(4C) and wt-αSyn(2SS) correspond to wt-αSyn with 4 Cys and 2 disulfides. The four cysteines in αSyn(4C) mutants were introduced at amino acid positions 9, 42, 69 and 89. (d) Among αSyn(2SS), there are 3 possible disulfide isomers. These isomers are distinguished by “a”, “b” and “c” as a second suffix. For instance, wt-αSyn(2SS)-a symbolizes isomer “a” of wt-αSyn(2SS). Isomers a, b and c have the disulfide connectivity of Cys⁹-Cys⁴²/Cys⁶⁹-Cys⁸⁹, Cys⁹-Cys⁶⁹/Cys⁴²-Cys⁸⁹, and Cys⁹-Cys⁸⁹/Cys⁴²-Cys⁶⁹, respectively.

Plasmid Construction:

Neuroblastoma cell line, SH-SY5Y, was cultured in DMEM/F12 medium (Gibco) containing 10% FBS (Hyclon) as described (Pan, et al., Neurosci. Lett., 377:106-9 (2005)). αSyn was obtained by PCR amplification of cDNAs that were generated by reverse transcription of total RNA isolated from SH-SY5Y using TRIzol reagent (Life Technol). The amplified products were cloned into pGEX5X-1 (Amersham Phamacia Biotech, Piscataway, N.J., U.S.A.) using XmaI and XhoI. The full length αSyn protein contains 140 amino acid residues. Two αSyn mutants that are involved in the pathogenesis of rare familiar PD are A53T and A30P, which have an Ala-to-Thr mutation at residue 53 and an Aal-to-Pro mutation at 30, respectively. The sequence of the constructs was verified by DNA sequencing.

Expression and Purification of GST-αSyn Fusion Proteins:

Expression of GST-αSyn fusion proteins in BL21 [F-ompT hsdSB(rB-mB-) gal dcm] cells (Stratagene) was induced with 0.5 mM isopropyl b-D-thiogalactoside (IPTG) for 4 h at room temperature. The cultures were collected by centrifugation and the bacterial pellets were re-suspended in Cellytic B bacterial cell lysis/extraction reagent (Sigma, B-3553) containing protease inhibitors (Sigma, P8849). The GST-αSyn fusion proteins were purified from crude cell lysates under non-denaturing conditions by selective binding to glutathione-Sepharose® 4B Beads (Amersham Pharmacia Biotech) as described by Choi, et al., Biotechnol. Appl. Biochem., 36:33-40 (2002). The GST-αSyn fusion proteins bound to beads were digested by Factor Xa (1/100, w/w, Amersham Pharmacia Biotech) in reaction buffer (100 mM NaCl, 50 mM Tris-HCl, 1 mM CaCl2, pH 8.0) at 25° C. for 16 h with gentle mixing following washes three times by PBS and one time reaction buffer. The cleaved proteins were further purified using HPLC. Purified αSyn proteins were lyophilized and stored at −80° C.

Results:

The pGEX expression system was utilized. In this expression system, wild-type and mutated αSyn proteins were expressed as GST-αSyn fusion proteins. Most of the expressed fusion proteins are present in the soluble state and almost undetectable in insoluble precipitates at the induction conditions of room temperature for 6 h. By selective binding to Glutathione Sepharose® beads, GST-αSyn fusion proteins were purified at high levels under non-denaturing conditions. A factor Xa proteolytic cleavage site has been engineered between GST and αSyn, allowing αSyn proteins to be efficiently cleaved for further HPLC purification.

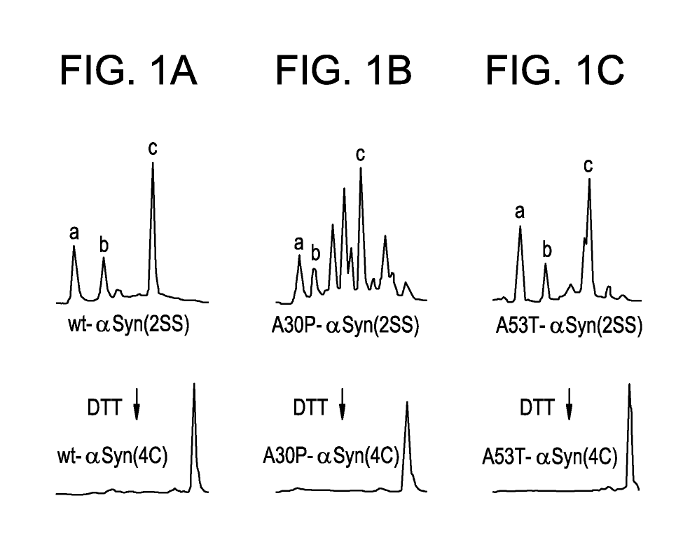

αSyn mutants obtained at this stage are already oxidized, but with different degrees of oxidation (FIG. 1). In the cases of wt-αSyn(4C) and A53T-αSyn(4C), oxidation is near completion, and 3 major 2-disulfide isomers (designated as “a”, “b” and “c”) are observed in each mutant. In the case of A30P-αSyn(4C), the oxidation of disulfide bonds is incomplete and the protein was recovered as a mixture of 1- and 2-disulfide isomers (there are 6 possible 1-disulfide isomers and 3 possible 2-disulfide isomers). These results are reproducible and therefore indicate that A30P-αSyn(4C) has a lower folding capability as compared to that of wt-αSyn(4C) and A53T-αSyn(4C) under identical conditions.

The three αSyn mutants were treated with denaturant and DTT to generate fully reduced species. They were then analyzed by HPLC and MALDI mass spectrometry. On HPLC (FIG. 1), wt-αSyn(4C), A30P-αSyn(4C) and A53T-αSyn(4C) exhibit a retention time almost indistinguishable from that of wt-αSyn; evidence of close similarity of their chemical property despite the insertion of four Ser/Ala [symbol] Cys mutations. The observed molecular mass of wt-αSyn (15230 obs/15229 exp), wt-αSyn(4C) (15325 obs/15325 exp), A30P-αSyn(4C) (15350 obs/15351 exp) and A53T-αSyn(4C) (15355 obs/15355 exp) are in agreement with their expected molecular weight.

### Example 2

### Oxidative Folding of wt-αSyn(4C), A30P-αSyn(4C) and A53T-αSyn(4C)

Materials and Methods:

Materials and methods are as described for Example 1 with the addition of those listed below.

Oxidative Folding of Ala/Ser [symbol] Cys Mutants of αSyn:

Purified mutant of αSyn was first reduced and denatured in Tris-HCl buffer (0.1M, pH 8.4) containing 6M GdmCl and 30 mM dithiothreitol, in order to generate the starting material for folding, αSyn(4C). The reaction was carried out for 90 minutes at 23° C. To initiate folding, the reduced αSyn mutant was passed through a PD-10 column (Sephadex®-25-Pharmacia) equilibrated in 0.1M Tris-HCl buffer (pH 8.4), diluted immediately with the same Tris-HCl buffer to a final protein concentration of 0.5 mg/ml. The oxidative folding was performed in this Tris-HCl buffer (0.1M, pH 8.4) alone or in the presence of CuSO₄ (2 μM). Folding intermediates were trapped in a time course manner by mixing aliquots of the sample with an equal volume of 4% aqueous trifluoroacetic acid and analyzed directly by reverse-phase HPLC.

HPLC Analysis for αSyn:

The purified isomers and mutants of αSyn were purified and analyzed by HPLC using the following conditions. Column was Zorbax® 300XB-C18, 250 mm×4.6 mm 5 μm. Buffer A was 0.1% TFA in water. Buffer B was 0.086% TFA in acetonitrile/water (9:1, by volume). The gradient of elution was 10% B to 70% B linear in 30 min. The flow rate was 0.5 ml/min. Column temperature was 23° C.

Characterization of Disulfide Structures of 2-Disulfide Isomers of wt-αSyn(2SS):

HPLC purified wt-αSyn(2SS)-a,b,c (15 μg) were digested with 1.5 μg of thermolysin (Sigma, P1512) in 30 μl of N-ethylmorpholine/acetate buffer (50 mM, pH 6.4), or with Glu-C in 30 μl of ammonium bicarbonate buffer (50 mM, pH 8.0). Digestion was carried out at 37° C. for 16 h. Peptides were then isolated by HPLC and analyzed by both MALDI mass spectrometry and Edman sequencing in order to identify peptide fragments containing disulfide bonds.

Analysis of Molecular Mass of Mutant and Isomers of αSyn:

The molecular mass of αSyn mutants and isomers, both unmodified and those modified with vinylpyridine and iodoacetic acid, were determined by MALDI mass spectrometer (Perkin-Elmer Voyager-DE STR) using 2,5-dihydroxybenzoic acid as matrix. Molecular mass of analyzed peptides were calibrated by the following standards. Bradykinin fragment (residues 1-7) (MH⁺ 757.3997); Synthetic peptide P14R (MH⁺ 1533.8582) and ACTH fragment (residues 18-39) (MH⁺ 2465.1989).

Results:

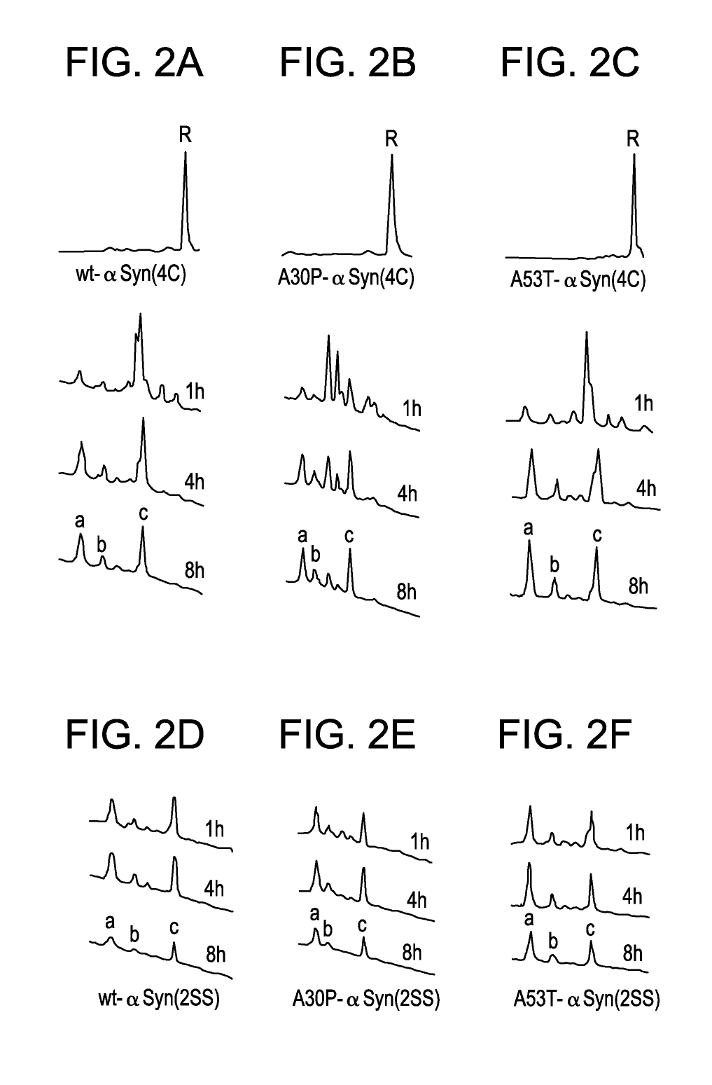

To further evaluate the properties of the three mutant proteins, systematic folding experiments of wt-αSyn(4C), A30P-αSyn(4C) and A53T-αSyn(4C) were conducted. Oxidative folding was carried out under the following conditions: (a) In the Tris-HCl buffer (0.1 M, pH 8.4) alone without redox agents. Under these conditions, air oxygen was responsible for the disulfide oxidation. Folding of wt-αSyn(4C) and A53T-αSyn(4C) completed within about 8 h, and each was shown to generate 3 isomers of αSyn(2SS) (FIGS. 2A and 2C). The folding of A30P-αSyn(4C) is slower than that of wt-αSyn(4C) and A53T-αSyn(4C) (FIG. 2B). (b) In the Tris-HCl buffer (0.1 M, pH 8.4) containing CuSO₄ (2 μM). Copper ions are known to catalyze air-oxidation of thiol groups to disulfide bonds (Ahmed, et al., J. Biol. Chem., 250:8477-82 (1975))). It was found that copper ions are useful catalysts to promote oxidative folding of αSyn(4C). The presence of 2 μM of CuSO₄ increases the folding rate of αSyn(4C) by approximately 4-fold (compare FIGS. 2A-2C to 2D-2F). However, the presence of copper ions also appears to accelerate the aggregation of folded αSyn(2SS). This is evident because in the presence of CuSO₄, the recovery of αSyn(2SS) isomers decreases significantly during extended period of folding, probably due to protein aggregation.

The report that Cu(II) concentration is elevated in the cerebrospinal fluid of PD patients (Pall, et al., Lancet, 2:238-241 (1987)) and that Cu(II) is the most effective ion in promoting oligomerization of αSyn synuclein (Paik, et al., Biochem. J., 340:821-828 (1999)) further suggest that copper ions might be a risk factor for the pathogenesis of PD. However, the copper concentrations most laboratories apply (0.5-5 mM) are far greater than those normally present in vivo. More recently, it was reported that copper at a physiologically relevant concentration is sufficient for accelerating αSyn aggregation (Rasia, et al., Proc. Natl. Acad. Sci. U.S.A., 102:4294-4299 (2005)). The copper concentration of 2 μM was intended to promote the oxidative folding of reduced αSyn(4C) mutants (Ahmed, et al., J. Biol. Chem., 250:8477-8482 (1975)). In this study, this concentration also effectively increased the kinetics of aggregation of folded (oxidized) 2-disulfide isomers of αSyn(2SS) (FIGS. 2D-2E). Alternatively, the copper enhanced oligomerization might be simply a consequence of increased folding kinetics.

The end-products of three folding experiments all comprise 3 major fractions of 2-disulfide isomers. This conclusion is substantiated by the observation that their modification with vinylpyridine affects neither their molecular mass nor their HPLC retention time; an evidence for the absence of free Cys. The three 2-disulfide isomers of wt-αSyn(2SS) were isolated and digested with thermolysin and Glu-C. Peptides were then isolated by HPLC and analyzed by MALDI mass spectrometry and Edman sequencing. Based on the known sequences of αSyn, the data of mass and sequence analysis allow identification of peptides containing the disulfide bonds and lead to the conclusion of disulfide pairings of wt-αSyn(2SS)-a, wt-αSyn(2SS)-b and wt-αSyn(2SS)-c, where isomers a, b and c have the disulfide connectivity of Cys⁹-Cys⁴²/Cys⁶⁹-Cys⁸⁹, Cys⁹-Cys⁶⁹/Cys⁴²-Cys⁸⁹, and Cys⁹-Cys¹⁹/Cys⁴²-Cys⁶⁹, respectively. The disulfide structures of 3 corresponding isomers of A30P-αSyn(2SS) and A53T-αSyn(2SS) have not been characterized, but most likely adopt the same disulfide pairing similar to that of wt-αSyn(2SS).

The existence of all 3 possible 2-disulfide isomers in significant and steady proportion in the mutants of wt-αSyn, A30P-αSyn and A53T-αSyn also supports the credence that αSyn is truly a natively unfolded protein consisting of diverse and defined conformational isomers. It is relevant to notice that wt-αSyn(2SS)-c is the most predominant isomer. Among the disulfide configuration of three isomers, wt-αSyn(2SS)-c represents the most compacted state. Its predominance thus implies that more compacted isomers account for the major structure in the natively unfolded state of wt-αSyn. In the cases of two αSyn variants, the concentrations of A30P-αSyn(2SS)-c and A53T-αSyn(2SS)-c decrease significantly, most likely due to the increase of less compacted isomers.

The findings that single mutations in wt-αSyn are linked to familial early-onset forms of PD points to a central role for the protein, in the etiology of the disease. Here it was found that A30P-αSyn(4C) has a much lower folding flexibility than that of wt-αSyn(4C) and A53T-αSyn(4C) under identical folding conditions (FIG. 2). The A30P mutant has been reported to exhibit a defective or reduced ability to bind lipid vesicles than does the wild-type protein (McLean, et al., J. Biol. Chem., 275:8812-8816 (2000); Perrin, et al., J. Biol. Chem., 275:34393-34398 (2000)). The formation of soluble oligomeric intermediates is also accelerated in the A30P mutant (Conway, et al., Proc. Natl. Acad. Sci. USA., 97:571-576 (2000)). A30P mutant favors the protein's self-aggregation capacity, as demonstrated in vitro (Conway, et al. Proc. Natl. Acad. Sci. USA., 97:571-576 (2000); Serpell, et al., Proc. Natl. Acad. Sci. U.S.A., 97:4897-4902 (2000)). Drosophila develops adult-onset motor dysfunctions that appear earlier in specimen expressing the A30P mutant than in those expressing WT or A53T αSyn (Feany, et al., Nature, 404, 394-398 (2000)).

These earlier observations may be explained by the unique folding behavior of A30P-αSyn(4C) demonstrated here. From the viewpoint of protein folding, αSyn appears to be a very dynamic molecule whose secondary structure depends on its environment. Therefore, the decreased folding flexibility of A30P-αSyn might weaken its dynamics and its physiological functions. On the other hand, the reduced conformational flexibility could also lead to the exposure of hydrophobic residues and makes A30P-αSyn naturally prone to forming insoluble aggregates.

### Example 3

### Alteration of Secondary Structure Accompanied by αSyn(4C) αSyn(2SS) Conversion

Materials and Methods:

Materials and methods are as described for Examples 1 and 2.

Results:

Human wt-αSyn is a natively unfolded protein and exhibits random coil structure at physiological condition. Upon lipid binding, wt-αSyn undergoes conformational change from random coil to α-helical structures (Davidson, et al., J. Biol. Chem., 272:9443-9449 (1998)). A53T mutation appears to have little effect on the αSyn-lipid interaction and subsequent conformational change (Bussell, et al., Biochemistry, 43:4810-4818 (2004)). The A30P mutation, however, results in either a defective or reduced ability of αSyn-lipid binding (Jo, et al., J. Mol. Biol., 315:799-807 (2002); Jensen, et al., J. Biol. Chem., 273:26292-26294 (1998)).

The reduced form of αSyn mutants, wt-αSyn(4C), A30P-αSyn(4C) and A53T-αSyn(4C), similar to that of wt-αSyn, all display structure of predominantly random coil (FIG. 3A). After oxidation and formation of two disulfide bonds, wt-αSyn(2SS) takes on a major portion of α-helical structure. In contrast, A30P-αSyn(2SS) and A 3T-αSyn(2SS) exhibit significant content of β-sheet structure (FIG. 3B). In essence, this wt-αSyn(4C) [symbol] wt-αSyn(2SS) conversion accompanied by random coil [symbol] α-helix transformation is akin to the structural change of wt-αSyn induced upon lipid binding. Both involve to some extent stabilization of natively unfolded structure of wt-αSyn. The puzzle is why similar conformational transformation does not apply to A53T-αSyn(2SS) and A30P-αSyn(2SS). Perceptibly, one can argue that the mode of stabilization of αSyn via lipid binding and introduced disulfide bonds may be very different. Satisfactory account of this discrepancy should also take into consideration that all 2-disulfide isomers of αSyn have 4 additional Ser/Ala [symbol] Cys mutations.

Nonetheless, the β-sheet content of A30P-αSyn(2SS) and A53T-βSyn(2SS) may facilitate their aggregation and this can explain their enhanced aggregative propensity as compared to that of wt-αSyn(2SS).

### Example 4

### The Isolated Isomers of wt-αSyn(2SS) Exhibit Dissimilar but Enhanced Aggregation Rate as Compared to wt-αSyn

Materials and Methods:

Materials and methods are as described for Examples 1-3 with the addition of those listed below.

Assay of Aggregation Rate of αSyn Isomers by Western Blot:

Aggregation of αSyn isomer was performed in PBS (pH 7.4) at room temperature or 37° C. The protein concentration was 20 μM. At different time points (0, 1, and 3 days), the samples were harvested for western blot assay. Equal quantity of the proteins from an equal volume of each fraction were boiled in sample buffer for 5 min, separated on an SDS-PAGE gel and transferred to nitrocellulose membrane. The membranes were blocked with 5% non-fat dry milk in PBST for 1 h at room temperature, probed with the primary antibodies in 5% non-fat dry milk overnight at 4° C., washed with PBS, and incubated with the an appropriate secondary antibody for 1 h at room temperature. The signals were detected using the enhanced chemiluminance assay (Amersham Life Science Inc., Arlington Height, Ill.), according to the manufacturer's instructions.

Assay of Fibrillation of αSyn Isomers by Fluorescence Measurement:

ThT fluorescence was recorded immediately following the addition of the aliquots of wt-Syn, wt-Syn(2SS)-a, wt-Syn(2SS)-b, wt-Syn(2SS)-c, and collective isomers of wt-Syn(2SS), A30P-Syn(2SS), and A53T-Syn(2SS) to the ThT mixture at different time points (0d, 1d, 3d and 7d). Measurements were performed at 37° C. and pH 7.4. Protein concentration was 1 mg/ml. ThT fluorescence was excited at 450 nm, and the emission wavelength was 482 nm.

Results:

The aggregation rate of a, b, c isomers of wt-αSyn(2SS) was compared to that of wt-αSyn. They were incubated in the PBS buffer (pH 7.4) at room temperature (22° C.) and 37° C. for different times (0, 1 and 3 days). Aggregated samples were analyzed by Western blot. The protein concentration was 20 μM. Rigorous control of the same protein concentration is essential to establish a convincing aggregation rate measurement, since higher protein concentration leads to a higher aggregation rate. The results show that under physiological conditions in vitro, all three isomers of wt-αSyn(2SS) display a faster aggregation rate than that of wt-αSyn, both at room temperature and at 37° C. Among the three isomers of wt-αSyn(2SS), the order of aggregation rate is c>a>b.

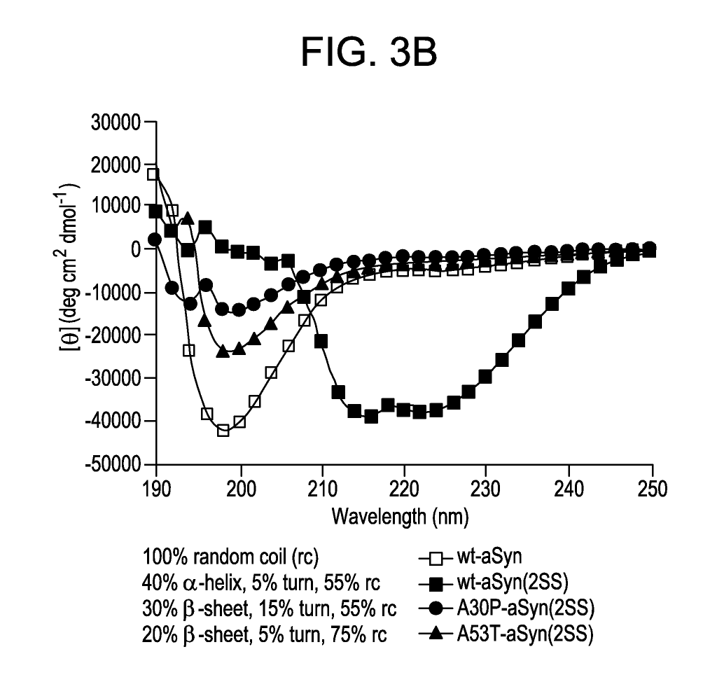

The differential and enhanced fibrillation was further verified by thioflavin T (ThT) fluorescence measurement. The results are shown in FIG. 4. The histological dye ThT is widely used for the detection of amyloid fibrils (Uversky, et al., J. Biol. Chem., 276, 10737-10744 (2001), Bartl, et al., Biophys. J., 68:104-110 (2001)). With the presence of fibrils, ThT gives rise to a new excitation maximum at 450 nm and enhanced emission at 482 nm, whereas naked ThT is essentially non-fluorescent at these wavelengths. ThT is supposed to bind specifically to the crossed β-sheet structure and the binding is independent of the primary structure of the protein. Only the multimeric fibrillar forms give significant fluorescence with ThT. The binding of ThT to α-Syn fibrils is therefore an effective method for the fibril formation assay. FIG. 4 shows the differences in the ThT fluorescence at different time points (1, 3 and 7 days) of α-Syn fibril formation. Consistent with the Western blot analysis, isomers a, b and c all exhibit higher ThT fluorescence, which suggests that the isomers have a different, but enhanced, propensity to fibrillate than wt-α-Syn. Among the isomers, wt-α-Syn(2SS)-c showed the highest propensity for fibril formation, which is consistent with the higher aggregation rate described above.

The results obtained here demonstrate that: (a) Different conformational isomers of wt-αSyn may exhibit different rate of aggregation and fibrillation; and (b) partial stabilization of the conformation of wt-αSyn (via two introduced disulfide bonds) may enhance the propensity of wt-αSyn aggregation and fibrillation. These data are consistent with the notion that under physiological conditions, human α-Syn exists as diverse conformational isomers which exhibit distinct propensity of aggregation and fibril formation.

### Example 5

### A30P-αSyn(2SS) has a Faster Aggregation Rate than A53T-αSyn(2SS) and wt-αSyn(2SS)

Materials and Methods:

Materials and methods are as described for the above examples.

Results:

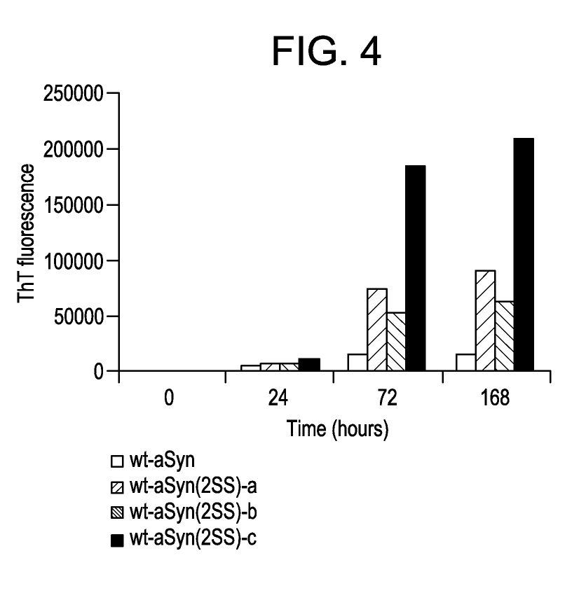

The aggregation rate of 2-disulfide isomers of wt, A30P and A53T in mixture form was further examined. Wt-αSyn(2SS), A30P-αSyn(2SS) and A53T-αSyn(2SS) were incubated for different time points (0, 1 and 3 days) for aggregation rate assay. It was found that even in the control (0d), there are strong bands at around 35 KD (dimer) and 70 KD (tetramer) in A30P-αSyn(2SS) isomers mixture, and after incubation (1d and 3d) at room temperature the oligomers increased significantly. In contrast, wt-αSyn(2SS) isomers mixture only had a insignificant band at around 35 KD at the time point of control and a comparatively weaker band at around 35 KD after 1 day or 3 days of incubation. The aggregation rate of A53T-αSyn(2SS) is between A30P-αSyn(2SS) and wt-αSyn(2SS). The data here suggest A30P-αSyn(2SS) has a much higher aggregation rate than A53T-αSyn(2SS), which in turn has a higher aggregation rate than wt-αSyn(2SS). The increased fibril formation was also confirmed by ThT fluorescence measurement and the results are shown in FIG. 5. A30P-αSyn(2SS) displays a higher tendency to fibrillate than A53T-αSyn(2SS) and wt-αSyn(2SS).

The faster aggregation and fibrillation rate of A30P-αSyn(2SS) mixture is probably due to its low folding flexibility described above. Higher aggregation rate of A30P-αSyn mutant has been consistently reported (Conway, et al., Proc. Natl. Acad. Sci. U.S.A., 97:571-6 (2000); Serpell, et al., Proc. Natl. Acad. Sci. U.S.A., 97:4897-4902 (2000)). It was also reported that PD-linked mutations accelerate αSyn aggregation (Narhi, et al., J. Biol. Chem., 274:9843-9846 (1999)). However, under their experimental conditions (protein concentration of 7 mg/ml, Tris-buffered saline at 37° C.), A53T-αSyn has a quicker aggregation rate than A30P-αSyn and WT-αSyn.

### Example 6

### Preparation of X-Isomers of Human α-Synuclein (2SS)

X-αSyn was prepared from WT-αSyn and two PD associated variants, A30P-αSyn and A53T-αSyn.

Materials and Methods:

Wt-αSyn does not comprise a disulfide bond. In order to generate conformational isomers of αSyn, a recombinant αSyn with 4 Ala/Ser [symbol] Cys mutations (at positions 9, 42, 69 and 89) was expressed and purified in reduced form as αSyn(4C), using the methods described above. αSyn(4C) was allowed to oxidize and fold in the Tris-HCl buffer containing CuSO₄ (1-5 μM) to form 2-disulfide isomers of X-αSyn.

Results

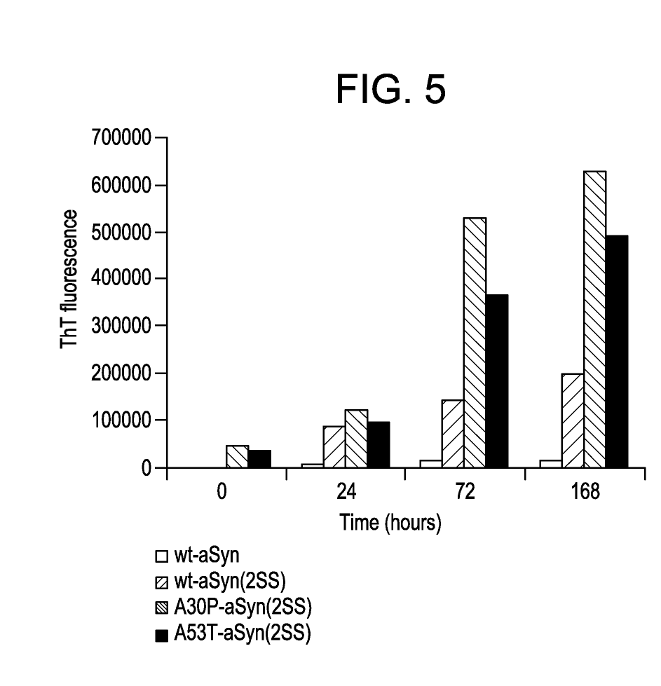

The expected isomers are shown in FIG. 9B. The HPLC analysis is shown in FIG. 6. The results demonstrate that efficient oxidation of αSyn(4C) requires the inclusion of CuSO₄ in the folding buffer and that diversity of X-αSyn can be amplified by altering AA [symbol] Cys mutation sites and the folding conditions.

### Example 7

### Expression and Oxidative Folding of α-Synuclein Containing 6 Cysteines

Materials and Methods:

Expression of α-synuclein containing 6 cysteines:

Vectors containing WT, A30P and A53T α-synuclein containing 6 cysteines were constructed and the proteins expressed as GST fusion proteins using the methodology generally described in Example 1. The 6 Ala/Ser to Cys mutations were introduced at positions 9, 42, 69, 89, 107 and 124. Among αSyn(3SS), there are 15 possible disulfide isomers. All other materials and methods are generally as described above with respect to Examples 1-6.

Results:

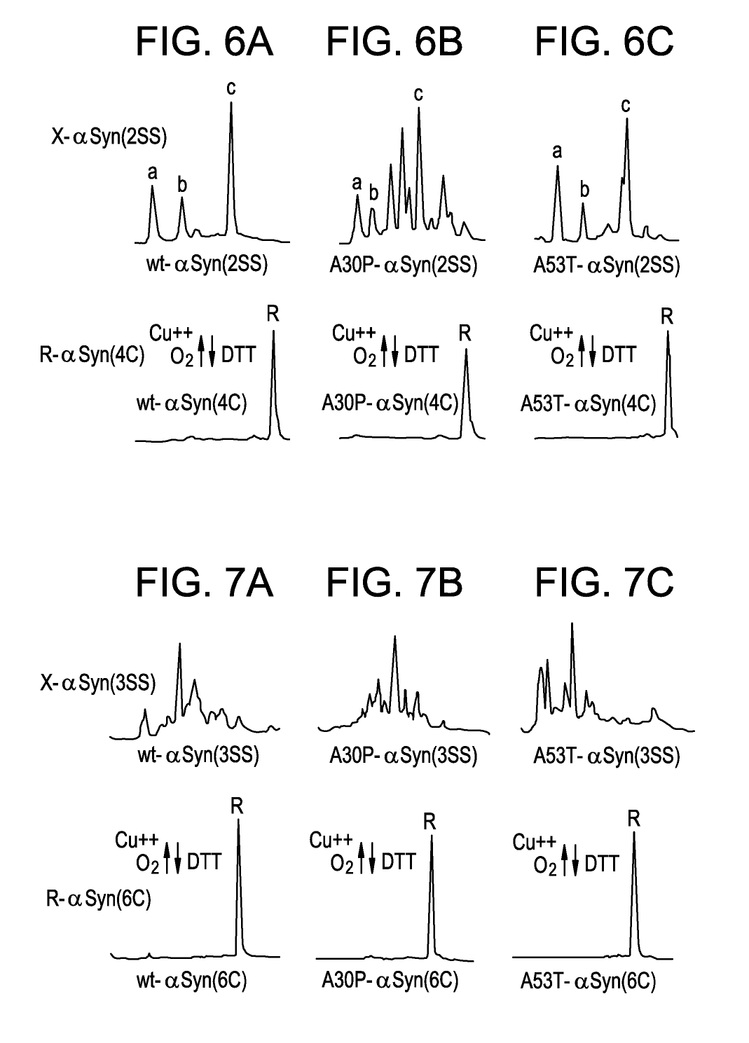

Six Ala/Ser [symbol] Cys mutations were introduced into WT-, A30P-, and A53T-αSyn at positions 9, 42, 69, 89, 107 and 124. The three αSyn mutants were expressed and purified in reduced form. They were then allowed to oxidize and fold in Tris-HCl buffer containing CuSO₄ (1-5 μM) to form 3-disulfide isomers of X-αSyn. Efficient oxidation of αSyn(6C) required the inclusion of CuSO₄ in the folding buffer. The isomers were then analyzed by HPLC. On HPLC (FIG. 7), wt-αSyn(6C), A30P-αSyn(6C) and A53T-αSyn(6C) exhibit a retention time almost indistinguishable from that of wt-αSyn; an evidence of close similarity of their chemical property despite the insertion of six Ser/Ala [symbol] Cys mutations. HPLC analysis of the oxidized forms of wt-αSyn(6C), A30P-αSyn(6C) and A53T-αSyn(6C) revealed a large number of conformational isomers exhibiting different retention times.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

We claim:

1. Conformational isomers of modified versions of αSyn.

2. The conformational isomers of claim 1 wherein the conformational isomers are produced by introducing cysteines into α-αSyn and scrambling the disulfide bonds to form stable and immunogenic isomers.

3. The conformational isomers of claim 2 wherein the isomers contain at least three introduced cysteines.

4. The conformational isomers of claim 3 wherein the isomers contain four introduced cysteines.

5. The conformational isomers of claim 3 wherein the isomers contain six introduced cysteines.

6. The conformational isomers of claim 1 wherein the αSyn is a variant αSyn associated with a disease.

7. The conformational isomer of claim 6 wherein the disease is Parkinson Disease.

8. The conformational isomers of claim 1 selected from the group consisting of 3-disulfide X-αSyn (3SS) produced by introducing 6 Cys mutations into the αSyn, 2-disulfide X-αSyn(2SS) produced by introducing 4 Cys mutations into the αSyn, A30P-αSyn, A53T-αSyn, X-A30P-αSyn(3SS), X-A53T-αSyn (3SS), X-A30P-αSyn(2SS), and X-A53T-αSyn (2SS).

9. The conformational isomer of claim 1 refolded in the presence of CuSO₄.

10. The conformational isomer of claim 9 refolded in buffer containing 1-5 μM CuSO₄.

11. A method of making antibodies to αSyn comprising immunizing an animal with any of the conformational isomers of claim 1, or making a derivative thereof.

12. The method of claim 11 wherein the derivative is a monoclonal, single chain or humanized antibody.

13. The antibodies produced by the method of claim 11.

14. A method for screening for αSyn comprising reacting the antibodies of claim 13 with a sample.

15. A method for screening for a disease using the conformational isomers of claim 1 as the target.

16. A method of treating a synucleinopathy comprising administering to an individual in need thereof the antibodies of claim 13.

17. A method of refolding a protein containing cysteines comprising refolding the denatured protein in a buffer containing CuSO₄.

18. The method of claim 17 wherein the buffer contains 1-5 μM CuSO₄.

19. The method of claim 18 wherein the protein is αSyn.
